(19) United States
(12) Patent Application Publication
(10) Pub. No.: US 2011/0020269 A1
(43) Pub. Date: Jan. 27, 2011

(54) METHODS AND COMPOSITIONS FOR MODIFYING T CELL IMMUNE RESPONSES AND INFLAMMATION

(75) Inventors: Terry B. Strom, Brookline, MA (US); Jeffrey Flier, Newton, MA (US); Marie Koulmanda, Brookline, MA (US)

Correspondence Address: FISH & RICHARDSON P.C. (BO), P.O. BOX 1022, MINNEAPOLIS, MN, 55440-1022, US

(73) Assignee: Beth Israel Deaconess Medical Center, Inc., Boston, MA (US)

(21) Appl. No.: 12/599,335

(22) PCT Filed: May 8, 2008
(86) PCT No.: PCT/US08/63133

§ 371 Date: Sep. 21, 2010

Related U.S. Application Data
(60) Provisional application No. 60/916,693, filed on May 8, 2007.

(51) Int. Cl. A61K 38/20; A61P 3/10; A61P 37/00; A61K 38/02; A61K 39/395; A61K 38/19

(52) U.S. Cl. 424/85.2; 514/1.1; 424/172.1; 424/85.1

(57) ABSTRACT

Methods and compositions for immune modulation are described herein. The compositions encompass pharmaceutical compositions that include a combination of agents. The first agent can selectively stimulate regulatory T cells or selectively inhibit inflammatory T cells and the second agent can reduce an inflammatory response in a tissue of a patient to whom the composition is administered. The second agent can reduce the expression or activity of a pro-inflammatory cytokine, promote the expression or activity of an anti-inflammatory cytokine, or both.

### CROSS-REFERENCE TO RELATED APPLICATIONS

This application claims the benefit of the filing date of U.S. provisional application No. 60/916,693, which was filed on May 8, 2007. For the purpose of any U.S. patent that may issue from the present application, the content of the prior provisional application is hereby incorporated by reference in its entirety.

### TECHNICAL FIELD

This invention relates to immunology, diabetes, and conditions associated with the immune system, such as autoimmune conditions.

### BACKGROUND

Cytokines direct newly activated T cells into distinct developmental pathways. For example, IL-12 stimulates the differentiation of T cells towards the Th1 phenotype. IL-4 promotes development to the Th2 phenotype. Differentiated T cell subsets possess characteristic cytokine expression profiles and effector functions. Th1 and Th17 cells orchestrate cell-mediated immune responses, while the effector functions of Th2 cells favor humoral immune responses. Th1 and Th17 subsets are linked to tissue-destructive immune responses, such as those connected with autoimmune diseases (Kolls and Linden, Immunity 21:467-376, 2004).

TGF-β plays a crucial role in T cell differentiation. In the absence of select pro-inflammatory cytokines, TGF-β directs newly activated CD4+ T cells into the tissue protective, FOXP3+ regulatory T cell phenotype. These FOXP3+ CD4+ T cells serve to protect, rather than destroy, tissues presenting the antigen to which they react. When TGF-β and certain pro-inflammatory cytokines (e.g., proteins primarily produced by mononuclear leukocyte inflammatory cells such as macrophages, monocytes, dendritic cells) are present within the milieu in which CD4+ T cells recognize antigen, the antigen stimulated T cells become Th17 cells (Bettelli et al., Nature 441(7090):235-238, 2006; Veldhoen et al., Immunity 24:179-189, 2006). Th17 cells are the most potent T cell mediators of injury of tissues bearing the stimulating antigen. Th17 cells produce interleukin 17 (IL-17), a cytokine that stimulates production of inflammatory cytokines by inflammatory cells. Commitment of cells to this lineage leads to a vicious cycle that links inflammation, pro-inflammatory Th17 cells and cytodestructive forms of T cell immunity.

### SUMMARY

The commitment of antigen stimulated T cells to a tissue protective FOXP3+ regulatory phenotype or to a tissue destructive, pro-inflammatory Th17 phenotype are reciprocally related. A milieu that fosters commitment to the tissue protective regulatory phenotype will deny CD4+ T cells entry into the tissue destructive Th17 phenotype and vice versa. A milieu dominated by Th17 cells produces severe T cell dependent tissue damage while a milieu dominated by regulatory T cells does not result in severe T cell dependent tissue damage. The ascendancy of protective, regulatory T cells can allow a patient to reduce the dosage of, if not completely stop, medications to treat conditions related to excessive or unwanted T cell activity. In some instances, tolerance enabled by the enduring ascendancy of regulatory T cells permits permanent cessation of immunosuppressive therapy. Thus, the key to achieving tolerance, e.g., in patients with autoimmunity or recipients of transplants, including transplants of allogeneic cells, lies in achieving an ascendancy of regulatory T cells over Th17 cells.

Breaking the vicious cycle connecting inflammation and pro-inflammatory T cells enables ascendancy of regulatory T cells and tolerance. Inflammation, if left unchecked, precludes commitment of CD4+ T cells into the regulatory phenotype while favoring commitment to the Th17 phenotype. If inflammation is incompletely dampened (e.g., such that there is residual expression of IL-6, TNFα, and/or IL-21), at least some Th17 cells will be present through prior commitment or leakiness. Hence, under many conditions, residual, albeit dampened, inflammation will enable commitment of antigen reactive T cells to the Th17 phenotype, which in turn will rekindle vigorous inflammation, allowing the cycle to persist and strengthen. In this scenario, severe immune mediated tissue injury, not tolerance, will be manifest.

To break this vicious cycle, we provide herein therapies that modify both T cell dependent tissue destructive forms of immunity and inflammation. The therapies achieve tolerance through combined utilization of two types of agents (e.g., one or more of each of the two types of agents). First are agents that foster commitment to, or selective retention of, antigen-specific regulatory T cells (as opposed to pro-inflammatory Th17 cells). Second are agents that foster creation of an environment in which expression of anti-inflammatory rather than pro-inflammatory molecules is favored. This combined therapy is a powerful means to hamper unwanted immune-mediated tissue destruction and produce tolerance.

Thus, the invention features, inter alia, combination therapeutics and therapies aimed at (1) shifting the balance from tissue destructive to tissue protective T cell immunity, and (2) dampening the expression or activity of proinflammatory molecules. Also featured is the use of these agents in the preparation of a medicament and/or the use of these agents in the preparation of a medicament for the treatment of diabetes, transplant rejection, autoimmune disease, and any other condition specified below. We may refer to the agents in category (1) as “first” agents, and the agents in category (2) as “second” agents.

In one aspect, the invention features pharmaceutical compositions including (1) a first agent that selectively stimulates regulatory T cells (e.g., FOXP3+ regulatory T cells) or selectively inhibits inflammatory T cells (e.g., Th17 cells. Th1 cells); and (2) a second agent that reduces an inflammatory response in a tissue of a patient to whom the composition is administered. The second agent may reduce the expression or activity of a pro-inflammatory cytokine, promote the expression or activity of an anti-inflammatory cytokine, or both. The compositions and methods may include more than one first agent and/or more than one second agent.

In general, the first agent is directed at T cells. Certain small molecule drugs that inhibit tissue destructive T cells more potently than regulatory T cells are useful as the first agent. One such agent is rapamycin. Calcineurin inhibitors and corticosteroids generally inhibit regulatory and inflammatory T cells with equal efficacy, and are not suitable as the first agent. However, under certain circumstances, calcineurin inhibitors and/or corticosteroids may be indicated for patients receiving the therapies received herein. Thus, the use of calcineurin inhibitors and/or corticosteroids may be used as an adjunct therapy; their use is not precluded by use of the agents or therapies described herein.

In some embodiments, the first agent is a polypeptide agent. Suitable polypeptide agents include anti-CD3 antibodies, particularly anti-CD3 antibodies that promote TGF-β expression, and related molecules (e.g., antigen binding fragments and biologically active derivatives thereof), and non-lytic anti-CD4 antibodies, and antigen binding fragments and biologically active derivatives thereof. Antibodies that destroy T cells without any selectivity (e.g., pan-T cell antibodies) are generally unsuitable as the first agent.

Agents that target molecules selectively expressed on, or by, Th17 cells are also suitable as the first agent. For example, the ligand of T cell immunoglobulin mucin 3 (TIM3), galectin 9, is expressed on proinflammatory T cells, such as Th17 cells, and inhibits their activity. Thus, agonists of TIM3 and/or galectin 9 are suitable as the first agent. Alternative, or in addition, agents (e.g., gene constructs) that result in overexpression of TIM3 and/or galectin 9 are suitable as the first agent. T cell immunoglobulin mucin 1 (TIM1) stimulates inflammatory T cells, hence TIM1 antagonists are useful agents (e.g., small molecules, anti-TIM1 antibodies, and nucleic acids (e.g., antisense oligonucleotides, aptamers, or siRNAs that inhibit TIM1 expression) are useful agents). Also useful are agents that inhibit the expression or activity of IL-17. For example, the present compositions can include antibodies that bind and neutralize IL-17, soluble IL-17 receptors, mutant IL-17 molecules that bind the IL-17R with high affinity and compete effectively with wild type IL-17 for the receptor, but fail to fully activate signal transduction through receptor, and IL-17-specific nucleic acids of the types just described in connection with TIM1.

IL-15 antagonists, as well as IL-2 agonists, are also contemplated as the first agent, alone or in combination. IL-15 antagonists include mutant IL-15 polypeptides that bind the IL-15R with high affinity and compete effectively with wild type IL-15 for the receptor, but fail to fully activate signal transduction through the IL-15R.

In some embodiments, the mutant IL-15 polypeptides (and other mutant and non-mutant cytokine compositions, as well as the AAT polypeptides described herein) further include an additional moiety that may increase the circulating half-life of the polypeptide. These moieties include an Fc region of an immunoglobulin molecule (e.g., an immunoglobulin of the G class). Soluble IL-15Rα polypeptides, or antibodies that specifically bind to IL-15 or the IL-15 receptor can also function as IL-15 antagonists.

IL-2 agonists include IL-2, fusion proteins with agonist activity, such as IL-2/Fc, mutants of IL-2 that retain the ability to bind and transduce a signal through the IL-2 receptor, and antibodies that specifically bind and agonize the IL-2 receptor (e.g., an antibody that specifically binds the α subunit of the IL-2 receptor).

In some embodiments, the pharmaceutical composition includes combinations of one or more of the above agents (e.g., an IL-15 antagonist, an IL-2 agonist, and rapamycin) and that triple combination may be further combined with and/or administered at the about the same time as AAT.

In general, the second agent inhibits a proinflammatory cytokine, either directly or indirectly. Direct inhibitors include agents that bind and neutralize the activity of a proinflammatory cytokine. Indirect inhibitors act, for example, by shifting expression profiles from proinflammatory (e.g., TNF-α, IFN-γ, GM-CSF, MIP-2, IL-6, IL-12, IL-1α, IL-1β, IL-21, and IL-23) to anti-inflammatory (e.g., IL-1rn, IL-4, IL-10, IL-11, IL-13, and TGF-β) cytokines in the patient.

AAT reduces expression of multiple proinflammatory cytokines. Because of its plieotropic effects on cytokines, AAT and agents that promote its expression or activity are particularly useful in the pharmaceutical compositions.

In some embodiments, the second agent is a cytoprotective agent such as an adenosine agonist or an agent that induce expression or activity of heme oxygenase-1 (HO-1) or A20. In other embodiments, the second agent is an adenylate cyclase agonist (e.g., prostaglandin), vitamin D, or an agonist thereof.

Immunoregulatory antigen presenting cells (APC) or regulatory T cells are also contemplated as the second agent.

In some embodiments, the second agent is an anti-inflammatory cytokine, or an agent that promotes its expression or activity. The anti-inflammatory cytokine can be selected from the group consisting of IL-1rn, IL-4, IL-10, IL-11, IL-13, and TGF-β. The cytokine can further include a moiety, such as the Fc region of an immunoglobulin, that increases its circulating half-life.

In other embodiments, the second agent is an agent that inhibits the expression or activity of an inflammatory cytokine, such as one of the following cytokines: TNF-α, IFN-γ, GM-CSF, MIP-2, IL-6, IL-12, IL-1α, IL-1β, IL-21, and IL-23. Exemplary inhibitors include antibodies and antigen binding fragments and derivatives thereof and soluble cytokine receptor molecules. These agents bind and neutralize the activity of the cytokine A soluble cytokine receptor can further include a moiety, such as the Fc region of an immunoglobulin, that increases its circulating half-life. The pharmaceutical composition can include one or more of the compounds described above as second agents. In some embodiments, TNF-α can be specifically excluded (i.e., the compositions can include any combination of the second agents just described with the exception of TNF-α).

The compositions, regardless of the precise active ingredients, can be formulated for administration by a particular route (e.g., intravenous, intramuscular, or subcutaneous administration).

Generally, the compositions described herein are useful for treating patients who would benefit from immune suppression (e.g., a patient who has, or is at risk for, an immune disease, particularly an autoimmune disease; a patient who has received, or is scheduled to receive, a transplant, e.g., a patient suffering from graft versus host disease (GVHD)).

Patients at risk for, or suffering from, a T cell mediated autoimmune disease particularly benefit from treatment with the compositions described herein. The autoimmune disease is, for example, type I diabetes, a rheumatic disease (e.g., rheumatoid arthritis, lupus erythematosus, Sjögren's syndrome, scleroderma, mixed connective tissue disease, dermatomyositis, polymyositis, Reiter's syndrome, and Behcet's disease), an autoimmune disease of the thyroid (e.g., Hashimoto's thyroiditis, or Graves' Disease), an autoimmune disease of the central nervous system (e.g., multiple sclerosis, myasthenia gravis, or encephalomyelitis), an ocular autoimmune disease (e.g., uveitis), an autoimmune disease of the gastrointestinal system (e.g., Crohn's disease, ulcerative colitis, inflammatory bowel disease, Celiac disease, Sprue), psoriasis, or Addison's disease.

In one embodiment, a composition described herein is used in a method of treating a patient at risk for, or diagnosed as having, Type 1 diabetes. In a related embodiment, the composition is used in a method of treating a patient who is insulin resistant (e.g., a patient who has Type 2 diabetes, is at risk of developing Type 2 diabetes, or has metabolic syndrome).

The compositions can also be used to treat a patient who has received a transplant of an organ, tissue, or cells, or who is scheduled to receive a transplant of an organ, tissue, or cells (i.e., the treatment can be given before or after the transplant; there is also no reason why the treatment could not be performed at essentially the same time as the transplant (i.e., while the patient is in the operating theater)).

Although the compositions of the invention can contain more than one agent, the methods of the invention are not limited to those in which the agents are formulated as a single composition or administered simultaneously. For example, a patient could receive a composition containing one or more of the compounds described as a suitable first agent before receiving a composition containing one or more of the compounds described as a second agent, or vice versa. Similarly, a patient could receive a composition containing one unique combination of a first and second agent, before receiving a composition containing another, different combination of a first and second agent. In some embodiments, the first and second agents will not be suitable for formulation as a single composition, and can be administered sequentially. In these embodiments, the first and second agent may be provided together in a package with the first and second agents in separate containers. The compositions of the invention, and methods for their use, are described further below.

In various embodiments, the present compositions may contain, as active ingredients, only one of each type of the “first” and “second” agents described below. In other embodiments, two or more such agents can be included, and in yet other embodiments, additional active agents can also be included. The pharmaceutically acceptable compositions can, of course, include any number of additional inert or inactive agents.

While the present methods are not limited to those that succeed due to any particular underlying cellular event(s), we have evidence that certain of the agents described herein, including AAT, can facilitate beta-cell regeneration and/or increase beta-cell mass. Residual beta-cells may be stimulated to proliferate. Other cell types, including less differentiated cells (e.g., stem cells or progenitor cells) may also proliferate and differentiate into more functional beta-cells. Accordingly, the methods of the invention include those for promoting beta-cell proliferation, differentiation, or regeneration.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a set of graphs depicting relative expression of cytotoxic T lymphocyte-, Th1- and pro-inflammatory cytokine-genes in IL-2/Fc+mIL-15/Fc+RPM (“Power Mix”) treated hosts. Gene expression profiles from pancreatic draining lymph notes harvested from newly diabetic (n=4, onset of T1DM within one week), old diabetic (n=4, diabetic more than 30 days), and Power Mix treated new onset diabetic mice (n=4, at day 50 following initiation of treatment) were analyzed. Expression of granzyme B (upper left panel), IFNγ (upper right panel), IL-1β (lower left panel), and TNFα (lower right panel) pro-inflammatory cytokine genes were analyzed and expressed relative to GAPDH expression.

FIGS. 2A-2F are a set photos of histological analyses of islets of spontaneous diabetic NOD mice at recent onset of disease and after 52 days after ending treatment with Power Mix (at least 70 days after onset). FIGS. 2A, 2B, and 2C show recent onset islets, in which most islets are atrophic with mainly glucagon-positive cells remaining after destruction of beta cells; a few residual islets are present (same islet in FIGS. 2B and 2C) with many beta cells but manifest insulitis-type invasion. FIGS. 2D, 2E, and 2F show islets 237 days after onset, after treatment and restoration of a euglycemic state; the islets with significant number of beta cells (same islet in FIGS. 2E and 2F) are surrounded by, but no longer invaded by, mononuclear leukocytes and have a more defined boundary of endocrine cells and are no longer degranulated. FIGS. 2A, 2B, 2D, and 2E show glucagon immunostaining FIGS. 2C and 2F show insulin immunostaining.

FIG. 3 is a graph depicting the results of Insulin tolerance tests (ITT) performed in age matched 1) spontaneous new onset diabetic NOD mice (NOD-sp); 2) the Power Mix treated spontaneous new onset NOD mice (NOD-sp/PM); 3) non-diabetic NOD mice. Results are expressed as percentage of initial blood glucose concentration.

FIG. 4 is a set of photos of immunoblot analyses of phosphorylated and non-phosphorylated insulin signaling proteins, insulin receptor (IR), insulin response substrate-1 (IRS-1), and PKB/Akt proteins, in skeletal muscle from normal, Power Mix treated, and diabetic mice.

FIGS. 5A-5D are photos of histological analyses of islets of spontaneous diabetic NOD mice analyzed at recent onset of diabetes (FIGS. 5A and 5B) and after treatment with AAT (100 days after onset) (FIGS. 5C and 5D). FIGS. 5A and 5C show pancreases that are immunostained for insulin, while FIGS. 5B and 5D are immunostained for glucagon. Magnification bars=50 μm.

FIGS. 6A-6D are graphs depicting RT-PCR results from Pancreatic lymph nodes comparing AAT treated NOD mice to chronic diabetic NOD mice. Results are expressed as intrasample target: GAPDH mRNA copy number ratio. *=0.05, **=0.01 (Mann-Whitney test was used for data analysis). IL-6=Interleukin-6, C3=Complement 3, INF-γ=Interferon gamma, Foxp-3=Fork head proteins P3, CRP+C-reactive protein, GBP=1 Guanylate nucleotide binding protein-1, IL-1β=Interleukin-1β, PAI-1=Plasminogen activator inhibitor type-1.

FIG. 7 is a graph depicting the results of insulin tolerance tests (ITT) performed in age matched 1) spontaneous new onset diabetic NOD mice (NOD-sp); 2) the AAT treated spontaneous new onset NOD mice (AAT); 3) non-diabetic NOD mice.

FIG. 8 is a set of graphs depicting results of immunoblot analysis of IR phosphorylation in 1) control non-diabetic NOD mice; 2) AAT treated NOD mice at 50 days; 3) acute diabetic NOD mice rendered euglycemic by delivery of insulin via a osmotic pump for 10 days; and 4) chronic diabetic NOD mice treated with conventional insulin therapy.

FIG. 9 is a set of graphs depicting results of RT-PCR results from fat comparing AAT treated NOD mice to chronic diabetic NOD mice. Results are expressed as intrasample target: GAPDH mRNA copy number ratio. *=0.05, **=0.01 (Mann-Whitney test was used for data analysis). Suppressor of cytokine signaling1 (SOCS1), Suppressor of cytokine signaling2 (SOCS2), and tissue necrosis factor alpha (TNFα).

FIG. 10 is a representation of an amino acid sequence encoding AAT (SEQ ID NO:1) and an AAT polypeptide sequence (SEQ ID NO:2).

FIG. 11 is a schematic representation of a non-cytolytic protein construct for human AAT (hAAT) fused to a human IgG Fc. The presence of two AAT polypeptides results in a dimer of sorts.

### DETAILED DESCRIPTION

T cell directed therapies that favor tolerance: This category of agents shifts the balance of T cells from tissue destructive to tissue protective phenotypes. Suitable T cell directed therapies allow for the ascendancy of regulatory T cells. Thus, the first agent may selectively stimulate regulatory T cells and/or selectively inhibit inflammatory T cells. Treatments that powerfully dampen both Th17 type destructive and regulatory T cell protective immunity with similar efficiency will not effectively promote tolerance (even when used in combination with anti-inflammatory treatment). Although agents that block both tissue destructive and tissue protective immunity with similar potency can be used to prevent immune mediated tissue injury, these agents fail to create a regulatory T cell dominant tolerant state, e.g., in a patient suffering from an autoimmune disease or experiencing transplant rejection.

In the present compositions and methods, the first agent can be: (a) rapamycin; (b) an anti-CD3 antibody or antigen binding fragment thereof; (c) a non-lytic anti-CD4 antibody or antigen binding fragment thereof; (d) a T cell immunoglobulin mucin 3 (TIM3) agonist; (e) a T cell immunoglobulin mucin 1 (TIM1) antagonist; (f) galectin 9 and agonists thereof; (g) an agent that selectively inhibits Th17 cells; (h) an agent that inhibits the expression or activity of interleukin 17 (IL-17); (i) an IL-15 antagonist; (j) an IL-2 agonist; or (i) a combination thereof. The combinations may be true combinations in the sense that they are physically contained within the same container or administered in combination by virtue of, for example, sequential administration or substantially simultaneous administration by different routes. For example, rapamycin may be delivered intramuscularly while an IL-15 antagonist and an IL-2 agonist are delivered intravenously.

Rapamycin is an example of a small molecule agent that effectively blocks tissue destructive T cell programs to a greater extent than it blocks regulatory T cell programs. As a consequence, rapamycin, albeit not sufficiently potent as a single drug, is useful in combination with other tolerance promoting drugs, including one or more of the first agents listed above. Rapamycin, as noted elsewhere herein, may be combined with and/or administered with an IL-15 antagonist and an IL-2 agonist.

Certain therapies are effective immunosuppressives but, because they block both tissue destructive and tissue protective immunity with equal potency, they are unreliable tolerance promoters. Therapies in this class are certain calcineurin inhibitors (e.g., cyclosporine, FK506) and corticosteroids.

IL-2 agonists (e.g., lytic IL-2/Fc) enhance activation induced cell death (AICD) of effector, but not regulatory T cells. An IL-2 agonist can inhibit an IL-2R. Accordingly, one can administer any agent that binds to and agonizes an IL-2R (e.g., an IL-2 per se or an IL-2 chimeric or fusion protein; see, e.g., Zheng et al., J. Immunol. 163:4041-4048, 1999).

IL-15 antagonists (e.g., mutant antagonist type IL-15/Fc) block proliferation and promotes passive cell death of activated effector T cells. The IL-15 antagonist may be one of the IL-15 mutant polypeptides described in U.S. Pat. No. 6,001,973. The mutant polypeptide can be at least or about 65% (e.g., at least or about 70%, 75%, 80%, 85%, 90%, 95%, 96%, 97%, 98% or 99% identical to a wild-type IL-15 (e.g., a wild-type human IL-15). The mutation can consist of a change in the number or content of amino acid residues. For example, the mutant IL-15 can have a greater or a lesser number of amino acid residues than wild-type IL-15. Alternatively, or in addition, the mutant polypeptide can contain a substitution of one or more amino acid residues that are present in the wild-type IL-15. The mutant IL-15 polypeptide can differ from wild-type IL-15 by the addition, deletion, or substitution of a single amino acid residue, for example, a substitution of the residue at position 149 or 156. Similarly, the mutant polypeptide can differ from wild-type by a substitution of two amino acid residues, for example, the residues at positions 156 and 149. For example, the mutant IL-15 polypeptide can differ from wild-type IL-15 by the substitution of aspartate for glutamine at residues 156 and 149 (as shown in FIGS. 14 and 15 of U.S. Pat. No. 6,001,973).

Where the first or second agent of the present compositions is a polypeptide, such as a cytokine, including a mutant interleukin as just described, or AAT, one may also use a therapeutically effective variant of the polypeptide. The variant may be a polypeptide that differs in sequence or in a post-translational feature such as glycosylation pattern.

Where a substitution is made to generate a polypeptide agent (e.g., a mutant IL-15), the substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine.

The mutations described above can be in the carboxy-terminal domain of the cytokine (e.g., in IL-15, a mutation can be made in the C-terminal domain, which is believed to bind the IL-2Rγsubunit; it is also possible that one or more mutations can be within the IL-2Rβ binding domain).

In a related aspect, any of the polypeptide agents can be chimeric polypeptides. For example, one could use a mutant IL-15 polypeptide as described above fused to one or more heterologous polypeptides (i.e., a polypeptide that is not IL-15 or a mutant thereof). The heterologous polypeptide can increase the circulating half-life of the chimeric polypeptide in vivo. The polypeptide that increases the circulating half-life may be a serum albumin, such as human serum albumin, or the Fc region of an immunoglobulin (e.g., the IgG subclass of antibodies that lacks the IgG heavy chain variable region). The Fc region may include a mutation that inhibits complement fixation and Fc receptor binding, or it may be lytic (i.e., able to bind complement or to lyse cells via another mechanism, such as antibody-dependent complement lysis (ADCC).

A person skilled in molecular biology can readily produce such molecules from, for example, an IgG2a-secreting hybridoma (e.g., HB129) or other eukaryotic cells or baculovirus systems. As noted and if desired, the Fc region can be mutated to inhibit its ability to fix complement and bind the Fc receptor. For murine IgG Fc, substitution of Ala residues for Glu 318, Lys 320, and Lys 322 renders the protein unable to direct ADCC. Substitution of Glu for Leu 235 inhibits the ability of the protein to bind the Fc receptor. Appropriate mutations for human IgG also are known (see, e.g., Morrison et al., The Immunologist, 2:119-124, 1994 and Brekke et al., The Immunologist, 2:125, 1994).

The “Fc region” can be a naturally-occurring or synthetic polypeptide that is homologous to the IgG C-terminal domain produced by digestion of IgG with papain. The polypeptide agents described herein can include the entire Fc region or a smaller portion that retains the ability to extend the circulating half-life of a chimeric polypeptide of which it is a part. In addition, and as noted, full-length or fragmented Fc regions can be variants of the wild-type molecule. That is, they can contain mutations that may or may not affect the function of the polypeptide; as described further below, native activity is not necessary or desired in all cases. In a preferred embodiment, the Fc region includes the hinge, CH2 and CH3 domains of human IgG1 or murine IgG2a.

The Fc region can be isolated from a naturally occurring source, recombinantly produced, or synthesized Oust as any polypeptide featured in the present invention can be). For example, an Fc region that is homologous to the IgG C-terminal domain can be produced by digestion of IgG with papain. The polypeptides of the invention can include the entire Fc region, or a smaller portion that retains the ability to lyse cells. In addition, full-length or fragmented Fc regions can be variants of the wild-type molecule. That is, they can contain mutations that may or may not affect the function of the polypeptide.

Chimeric polypeptides can be constructed using no more than conventional molecular biological techniques, which are well within the ability of those of ordinary skill in the art to perform.

As used herein, the terms “protein” and “polypeptide” both refer to any chain of amino acid residues, regardless of length or post-translational modification (e.g., glycosylation or phosphorylation).

Examples biologic therapies that fail to discriminate between regulatory and Th17 cells include pan-T cell or anti-CD4 destructive monoclonal or polyclonal antibodies, antagonist type anti-Tim3 or anti-Tim3 ligand mAbs, agonist type anti-Tim-1 mAbs, Tim4Ig, and certain anti-OX40 mAbs.

Agents that shift the balance of inflammation from expression of pro-inflammatory (e.g., IL-1, IL-6, TNFα, IL-21) to anti-inflammatory (TGF-α, IL1RA, IL-10) cytokines: Shifting the balance from pro- to anti-inflammatory responses aids in the formation of tolerizing tissue protective T cell directed immunity. Agents that block production of pro-, but not anti-, inflammatory cytokines are useful as the second agent. A potent example of this type of agent is alpha 1-antitrypsin (A1AT or AAT).

AAT is one of the main components of blood protein. It is synthesized in the liver and secreted into the plasma. The A1AT enzyme acts as an inhibitor of various proteases, but its main target is elastase. In the absence of A1AT, elastase is free to break down elastin which contributes to the elasticity of the lungs and result in respiratory complications such as emphysema leading finally to chronic obstructive pulmonary disease (COPD).

AAT is currently commercially available, and such formulations can be used in the present combinations and methods. Baxter International, Inc. markets AAT as Aralast™ for the treatment of chronic augmentation therapy in patients with hereditary emphysema. AAT can be prepared from large pools of human plasma by using the Cohn-Oncley cold alcohol fractionation process, followed by purification steps including polyethylene glycol and zinc chloride precipitation and ion exchange chromatography. Because the metabolic half-life of Aralast is 5.9 days, we expect dosing approximately once weekly (e.g., with a dosage of 60 mg/kg body weight).

A1AT is a single glycoprotein consisting of 394 amino acids in the mature form. There are three N-linked glycosylation sites, mainly equipped with so-called diantennary N-glycans. These glycans carry different amounts of negative-charged sialic acids which cause the heterogeneity in normal A1AT.

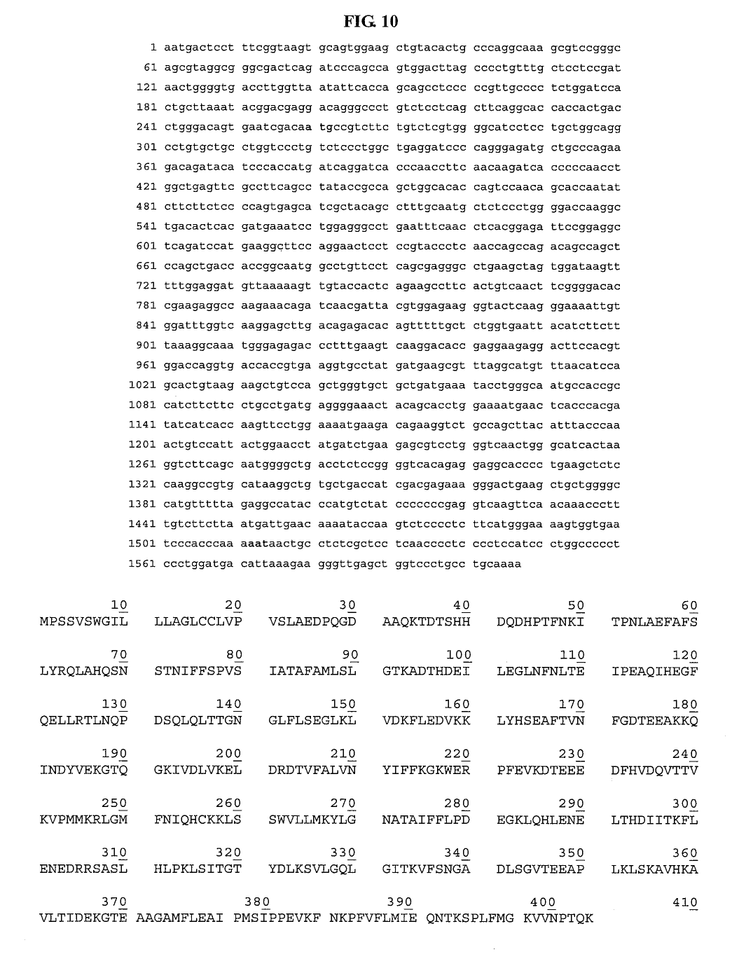

As shown in FIG. 10, AAT can be encoded by 1254 bp of nucleotide sequence with a starting methionine encoded beginning at position 260 and a stop beginning at position 1514. The protein sequence has been translated into 418 aa with MW 46,737 and the PI is 5.37. Further analysis reveal four potential sites to be N-glycosylated and these four asparagines residues (N) are located at N70, N107, N271 and N414.

To clone and express an AAT polypeptide one can, for example, subclone cDNA of AAT into the pFUFC vector from InvivoGen to create an AAT-IgG-Fc fusion protein; the portion A1AT-IgG-Fc portion of this plasmid can be cloned into the UCOE expression vector from Millipore; and stable human A1AT expressed in CHO cells can be screened by ELISA. Large amounts of AAT fusion protein can be produced by the WAVE bioreactor system, and purification can be achieved with proteinA affinity columns. We choose the UCOE (ubiquitous chromatin opening element) expression system because it gives major improvements in gene expression in stably-transfected mammalian cells.

AAT activity can be measured in terms of the inhibitory capacity of AAT toward trypsin. Microplates can be coated with 1% of FBS and incubated for one hour. After a wash (3×) with H₂O, various concentrations of AAT can be incubated with a fixed amount of trypsin for 20 minutes at 37° C. in a 200 μl final reaction volume. A chromogenic substrate (L-pyroglutamylglycyl-L-arginine, p-nitroanilide hydrochloride) can then be added to the plate and incubation continued for about 5 minutes at room temperature. The reaction can be stopped with 50 μl of 50% acetic acid. Absorbance can be read at 400 nm in a microplate reader.

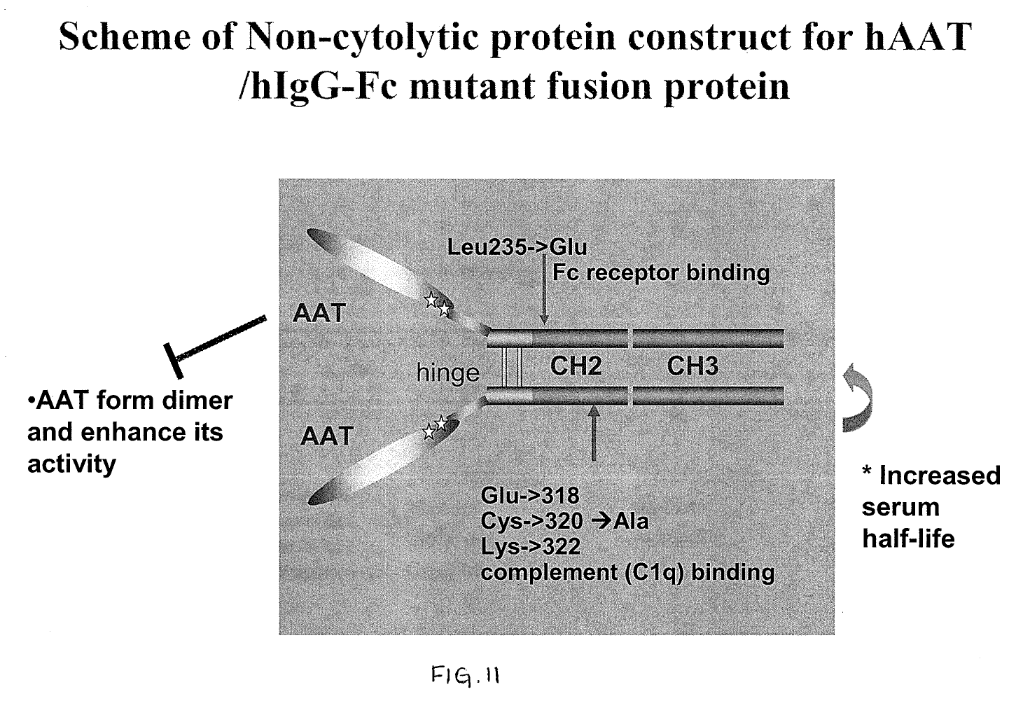

As shown in FIG. 11 the AAT agent can be fused to an Fc region of an IgG, in which case two AAT polypeptides (or therapeutically active variants thereof) are included in a single molecule.

Because AAT dampens expression of multiple pro-inflammatory cytokines while not hindering or even enhancing expression of the TGF-β and IL-1RA anti-inflammatory cytokines, other agents that neutralize, antagonize, block production or intracellular signaling of IL-6 and/or TNFα and/or IL-1β and/or IL-21 and/or IL-23 without impairing or enhancing expression of anti-inflammatory cytokines (e.g. TGF-β, IL-10, and IL1RA) will synergize with the tolerance-promoting T cell directed regimens described herein. Antagonist type anti-receptor antibodies or mutant antagonist type cytokines are examples of agents in this category. Agents that selectively block intracellular signaling pathways spawned by pro-, but not anti-, inflammatory cytokines are also useful as second agents. Compounds that exert cytoprotective properties including A20, HO-1 inducers, and adenosine agonists will have directionally similar effects, albeit perhaps less potent, to that exerted by AAT.

If the shift from a pro- to anti-inflammatory state is complete, compatible T cell directed strategies may be reduced or eliminated from a patient's treatment regimen. In some embodiments, the combinations will have super additive, beneficial effects. An example of a super additive combination is the combined use of: an IL-2 agonist, an IL-15 antagonist, rapamycin, and either Aralast™ (human AAT), or IL-10/Ig, a fusion protein which has an enhanced circulating half life relative to wild type IL-10. In a monkey allogeneic islet cell transplant model, these combined therapies, used short term (28 days), enabled superb early transplant function despite use of a remarkably small mass of islets for transplantation and freedom from rejection.

The sections below more generally describe the first and second agents and methods of making them.

In some embodiments, the first agent and/or the second agent is an antibody or fragment thereof, such as an antibody that specifically binds CD3, CD4, TIM3, TIM1, or a cytokine. Antibodies and fragments thereof are useful in that they interfere with pro-inflammatory effector functions directly (e.g., by blocking receptor-ligand interactions, such as IL-17 binding to IL-17 receptors), or indirectly (e.g., by inhibiting a moiety in the pathway that is required for a pro-inflammatory component, such as TNFα, to affect cellular processes).

An antibody that selectively binds to the target of interest and is useful as an agent of the present compositions can be a whole antibody, including a whole human, humanized, or chimeric antibody, or an antibody fragment or subfragment thereof. The antibody can be a whole immunoglobulin of any class (e.g., IgG, IgM, IgA, IgD, and IgE), a chimeric antibody, a humanized antibody, or a hybrid antibody with dual or multiple antigen or epitope specificities (e.g., a bispecific antibody). The fragments can be, for example, F(ab)₂, Fab′, Fab, and the like, including hybrid fragments. In addition to classic monovalent antibody fragments such as Fab and scFv (i.e., single chain antibodies), engineered variants such as diabodies, triabodies, minibodies, and single-domain antibodies can also be used. The antibody can further be any immunoglobulin or any natural, synthetic or genetically engineered protein that acts like an antibody by binding to the target to form a complex. In particular, Fab molecules can be expressed and assembled in a genetically transformed host like E. coli. A lambda vector system is available to express a population of Fab's with a potential diversity equal to or exceeding that of subject generating the predecessor antibody (see Huse et al., Science 246:1275-1281, 1989). The antibody can be a monoclonal antibody.

Examples of commercially available therapeutic antibodies that bind the targets of interest are anti-TNFα antibodies, adalimumab (Humira™), infliximab (Remicade™), CDP571 (a humanized monoclonal anti-TNFα antibody) and anti-CD3 antibodies (Orthoclone OKT3®).

Methods of making and using antibodies are now well-known in the art (see, e.g., Antibodies, Ed Harlow and David Lane (Eds.), CSHL Press, Cold Spring Harbor, N.Y., 1988; Using Antibodies, Ed Harlow and David Lane (Eds.), CSHL Press, Cold Spring Harbor, N.Y., 1998), and those techniques can be applied to generate an antibody useful in the present compositions and methods.

Alternatively, or in addition, an agent can be a soluble cytokine or cytokine receptor. The cytokine or receptor can be joined to an immunoglobulin molecule or a portion thereof (e.g., an Fc region (e.g., an Fc region of an IgG molecule)). One example of a soluble receptor antagonist can be etanercept (Enbrel™). Etanercept is a recombinant fusion protein consisting of two soluble TNF receptors joined by the Fc fragment of a human IgG1 molecule. Etanercept is currently approved only for rheumatoid arthritis and is provided as a subcutaneous injection of 25 mg given twice a week. This regimen produces peak blood levels in an average of 72 hours.

A soluble receptor or cytokine agent can include a full-length, soluble form of the receptor or cytokine, or a portion or other mutant thereof that retains sufficient activity to reduce activity of the target of interest to a clinically useful extent. For example, the antagonist can be, or can include, the previously identified C-terminal truncated form of the soluble human TNF receptor type I. The receptor or cytokine can be PEGylated (see, e.g., Edwards et al., Adv. Drug. Delivery Res. 55:1315-1336, 2003).

Other useful agents which function as inhibitors include agents that selectively inhibit expression (e.g., expression of a pro-inflammatory cytokine), such as RNA molecules that mediate RNAi (e.g., a TNFα selective siRNA or shRNA) and antisense oligonucleotides. More specifically, one can administer a molecule that mediates RNAi (e.g., a short interfering nucleic acid (siNA), a short interfering RNA (siRNA), a double-stranded RNA (dsRNA), or a short hairpin RNA (shRNA) as described in published U.S. Patent Application No. 20050227935, the contents of which are incorporated herein by reference in their entirety.

Useful agents also include those that selectively modulate (e g., inhibit) a moiety within the signaling pathway of a target molecule (e.g., a cytokine), such as inhibitors of a TNFα signaling pathway. A known and useful IL-1 antagonist, which may be incorporated in the present compositions and methods is anakinra (Kineret™).

Procedures for Screening Agents that Inhibit the Immune Response: Candidate agents can be tested using in vitro assays or any of the following in vivo assays, to determine which particular agents most effectively function in combination to bring about immune suppression. For example, one can test one or more of the agents that block IL-17 or the differentiation of Th17 cells in combination with one or more of the agents that block inflammatory mechanism. These in vivo assays represent only some of the routine ways in which one of ordinary skill in the art could further test the efficacy of agents of the invention. They were selected for inclusion here because of their relevance to the variety of clinical conditions amenable to treatment with agents that bring about immune suppression and tolerance. For example, the assays are relevant to organ transplantation, immune disease, particularly autoimmune disease, graft versus host disease and cancers (e.g., cancers of the immune system).

Transplantation Paradigms: To determine whether a combination of agents of the invention achieves immune suppression, the combination can be administered (either directly, by gene-based therapy, or by cell-based therapy) in the context of well-established transplantation paradigms.

Agents of the invention, nucleic acid molecules encoding them (or that hybridize with and thereby inhibit them), can be systemically or locally administered by standard means to any conventional laboratory animal, such as a rat, mouse, rabbit, guinea pig, or dog, before an allogeneic or xenogeneic skin graft, organ transplant, or cell implantation is performed on the animal. Strains of mice such as C57B1-10, B10.BR, and B10.AKM (Jackson Laboratory, Bar Harbor, Me.), which have the same genetic background but are mismatched for the H-2 locus, are well suited for assessing various organ grafts.

Heart Transplantation: A method for performing cardiac grafts by anastomosis of the donor heart to the great vessels in the abdomen of the host was first published by Ono et al. (J. Thorac. Cardiovasc. Surg. 57:225, 1969; see also Corry et al., Transplantation 16:343, 1973). By way of this surgical procedure, the aorta of a donor heart is anastomosed to the abdominal aorta of the host, and the pulmonary artery of the donor heart is anastomosed to the adjacent vena cava using standard microvascular techniques. Once the heart is grafted in place and warmed to 37° C. with Ringer's lactate solution, normal sinus rhythm will resume. Function of the transplanted heart can be assessed frequently by palpation of ventricular contractions through the abdominal wall. Rejection is defined as the cessation of myocardial contractions. Agents of the invention would be considered effective in reducing organ rejection if hosts that received these agents experienced a longer period of engraftment of the donor heart than did untreated hosts.

Skin Grafting: The effectiveness of various combinations of the agents of the invention can also be assessed following a skin graft. To perform skin grafts on a rodent, a donor animal is anesthetized and the full thickness skin is removed from a part of the tail. The recipient animal is also anesthetized, and a graft bed is prepared by removing a patch of skin from the shaved flank. Generally, the patch is approximately 0.5×0.5 cm. The skin from the donor is shaped to fit the graft bed, positioned, covered with gauze, and bandaged. The grafts can be inspected daily beginning on the sixth post-operative day, and are considered rejected when more than half of the transplanted epithelium appears to be non-viable. Agents of the invention would be considered effective in reducing skin graft rejection if hosts that received these agents experienced a longer period of engraftment of the donor skin than did untreated hosts.

Islet Allograft Model: DBA/2J islet cell allografts can be transplanted into rodents, such as 6-8 week-old B6 AF1 mice rendered diabetic by a single intraperitoneal injection of streptozotocin (225 mg/kg; Sigma Chemical Co., St. Louis, Mo.). As a control, syngeneic islet cell grafts can be transplanted into diabetic mice. Islet cell transplantation can be performed by following published protocols (for example, see Gotoh et al., Transplantation 42:387, 1986). Briefly, donor pancreata are perfused in situ with type IV collagenase (2 mg/ml; Worthington Biochemical Corp., Freehold, N.J.). After a 40-minute digestion period at 37° C., the islets are isolated on a discontinuous Ficoll gradient. Subsequently, 300-400 islets are transplanted under the renal capsule of each recipient. Allograft function can be followed by serial blood glucose measurements (Accu-Check III™; Boehringer, Mannheim, Germany). Primary graft function is defined as a blood glucose level under 11.1 mmol/l on day 3 post-transplantation, and graft rejection is defined as a rise in blood glucose exceeding 16.5 mmol/l (on each of at least 2 successive days) following a period of primary graft function.

Models of Autoimmune Disease: Models of autoimmune disease provide another means to assess combinations of the agents of the invention in vivo. These models are well known to those of ordinary skill in the art and can be used to determine whether a given combination of agents would be therapeutically useful in treating a specific autoimmune disease when delivered either directly, via genetic therapy, or via cell-based therapies.

Autoimmune diseases that have been modeled in animals include rheumatic diseases, such as rheumatoid arthritis and systemic lupus erythematosus (SLE), type I diabetes, and autoimmune diseases of the thyroid, gut, and central nervous system. For example, animal models of SLE include MRL mice, BXSB mice, and NZB mice and their F₁ hybrids. These animals can be crossed in order to study particular aspects of the rheumatic disease process; progeny of the NZB strain develop severe lupus glomerulonephritis when crossed with NZW mice (Bielschowsky et al., Proc. Univ. Otago Med. Sch. 37:9, 1959; see also Fundamental Immunology, Paul, Ed., Raven Press, New York, N.Y., 1989). Similarly, a shift to lethal nephritis is seen in the progeny of NBZ X SWR matings (Data et al., Nature 263:412, 1976). The histological appearance of renal lesions in SNF₁ mice has been well characterized (Eastcott et al., J. Immunol. 131:2232, 1983; see also Fundamental Immunology, supra). Therefore, the general health of the animal as well as the histological appearance of renal tissue can be used to determine whether the administration of agents can effectively suppress the immune response in an animal model of SLE.

Animal models of intestinal inflammation are described, for example, by Elliott et al. (Elliott et al., 1998, Inflammatory Bowel Disease and Celiac Disease. In: The Autoimmune Diseases, Third ed., N. R. Rose and I. R. MacKay, eds. Academic Press, San Diego, Calif.). Some mice with genetically engineered gene deletions develop chronic bowel inflammation similar to IBD. See, e.g., Elson et al., Gastroenterology 109:1344, 1995; Berg et al., J. of Clin. Investigation 98:1010,1996; Ludviksson et al., J. Immunol. 158:104,1997; and Mombaerts et al., Cell 75:274, 1993). These include mutant mice with targeted deletions for IL-2, IL-10, MHC class II or TCR genes among others.

One of the MRL strains of mice that develops SLE, MRL-lpr/lpr, also develops a form of arthritis that resembles rheumatoid arthritis in humans (Theofilopoulos et al., Adv. Immunol. 37:269, 1985). Alternatively, an experimental arthritis can be induced in rodents by injecting rat type II collagen (2 mg/ml) mixed 1:1 in Freund's complete adjuvant (100 μl total) into the base of the tail. Arthritis develops 2-3 weeks after immunization. The effectiveness of a candidate treatment is assessed by following the disease symptoms during the subsequent 2 weeks, as described by Chernajovsky et al. (Gene Therapy 2:731-735, 1995). Lesser symptoms, compared to control, indicate that the combined agents of the invention, and the nucleic acid molecules that encode them, function as immunosuppressants and are therefore useful in the treatment of immune disease, particularly autoimmune disease.

The ability of various combinations of agents to suppress the immune response in the case of Type I diabetes can be tested in the NOD (non-obese diabetic) mouse model discussed in the Examples, below, or in the BB rat strain, which was developed from a commercial colony of Wistar rats at the Bio-Breeding Laboratories in Ottawa. These rats spontaneously develop autoantibodies against islet cells and insulin, just as occurs with human Type I diabetes.

Autoimmune diseases of the thyroid have been modeled in the chicken. Obese strain (OS) chickens consistently develop spontaneous autoimmune thyroiditis resembling Hashimoto's disease (Cole et al., Science 160:1357, 1968). Approximately 15% of these birds produce autoantibodies to parietal cells of the stomach, just as in the human counterpart of autoimmune thyroiditis. The manifestations of the disease in OS chickens, which could be monitored in the course of any treatment regime, include body size, fat deposit, serum lipids, cold sensitivity, and infertility.

Models of autoimmune disease in the central nervous system (CNS) can also be experimentally induced. An inflammation of the CNS, which leads to paralysis, can be induced by a single injection of brain or spinal cord tissue with adjuvant in many different laboratory animals, including rodents and primates. This model, referred to as experimental allergic encephalomyelitis (EAE) is T cell mediated. Similarly, experimentally induced myasthenia gravis can be produced by a single injection of acetylcholine receptor with adjuvants (Lennon et al., Ann. N.Y. Acad. Sci. 274:283, 1976).

Nucleic Acid Molecules That Encode Agents of the Invention: Polypeptide agents of the invention, including those that are fusion proteins (e.g., cytokine/Fc fusions, such as the mutant IL-15/Fc and IL-2/Fc molecules discussed herein) can not only be obtained by expression of a nucleic acid molecule in a suitable eukaryotic or prokaryotic expression system in vitro and subsequent purification of the polypeptide agent, but can also be administered to a patient by way of a suitable gene therapeutic expression vector encoding a nucleic acid molecule. Furthermore a nucleic acid can be introduced into a cell of a graft prior to transplantation of the graft. Thus, nucleic acid molecules encoding the agents described above are within the scope of the invention. Just as polypeptides of the invention can be described in terms of their identity with wild-type polypeptides, the nucleic acid molecules encoding them will necessarily have a certain identity with those that encode the corresponding wild-type polypeptides. For example, the nucleic acid molecule encoding a cytokine polypeptide can be at least 65%, preferably at least 75%, more preferably at least 85%, and most preferably at least 95% (e.g., 96%, 97%, 98%, or 99%) identical to the nucleic acid encoding wild-type cytokine For nucleic acids, the length of the sequences compared will generally be at least 50 nucleotides, preferably at least 60 nucleotides, more preferably at least 75 nucleotides, and most preferably 110 nucleotides.

The nucleic acid molecules that encode agents of the invention can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules of the invention may be referred to as “isolated” when they are separated from either the 5′ or the 3′ coding sequence with which they are immediately contiguous in the naturally occurring genome of an organism. Thus, the nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced by in vitro transcription.

The isolated nucleic acid molecules of the invention can include fragments not found as such in the natural state. Thus, the invention encompasses recombinant molecules, such as those in which a nucleic acid sequence is incorporated into a vector (for example, a plasmid or viral vector) or into the genome of a heterologous cell (or the genome of a homologous cell, at a position other than the natural chromosomal location).

As described above, agents of the invention can be fusion proteins. In addition to, or in place of, the heterologous polypeptides described above, a nucleic acid molecule encoding an agent of the invention can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, one of ordinary skill in the art will be aware of additional useful reagents, for example, of additional sequences that can serve the function of a marker or reporter.

The nucleic acid molecules described above can be contained within a vector that is capable of directing their expression in, for example, a cell that has been transduced with the vector. Accordingly, in addition to polypeptide agents, expression vectors containing a nucleic acid molecule encoding those agents and cells transfected with those vectors are among the preferred embodiments.

Vectors suitable for use in the present invention include T7-based vectors for use in bacteria (see, e.g., Rosenberg et al., Gene 56:125, 1987), the pMSXND expression vector for use in mammalian cells (Lee and Nathans, J. Biol. Chem. 263:3521, 1988), yeast expression systems, such as Pichia pastoris (for example the PICZ family of expression vectors from Invitrogen, Carlsbad, Calif.) and baculovirus-derived vectors (for example the expression vector pBacPAK9 from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. One of ordinary skill in the art is well aware of numerous promoters and other regulatory elements that can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 resistance to cells in which it is expressed, and thus permits phenotypic selection of the transfected cells. Other feasible selectable marker genes allowing for phenotypic selection of cells include various fluorescent proteins, e.g. green fluorescent protein (GFP) and variants thereof. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, e.g., Gluzman (Ed.), Eukaryotic Viral Vectors, CSH Laboratory Press, Cold Spring Harbor, N.Y.).

Prokaryotic or eukaryotic cells that contain a nucleic acid molecule that encodes an agent of the invention and express the protein encoded in that nucleic acid molecule in vitro are also features of the invention. A cell of the invention is a transfected cell, i.e., a cell into which a nucleic acid molecule, for example a nucleic acid molecule encoding a polypeptide, has been introduced by means of recombinant DNA techniques. The progeny of such a cell are also considered within the scope of the invention. The precise components of the expression system are not critical. For example, a polypeptide can be produced in a prokaryotic host, such as the bacterium E. coli, or in a eukaryotic host, such as an insect cell (for example, Sf21 cells), or mammalian cells (e.g., COS cells, CHO cells, 293 cells, NIH 3T3 cells, or HeLa cells). These cells are available from many sources, including the American Type Culture Collection (Manassas, Va.). In selecting an expression system, it matters only that the components are compatible with one another. One of ordinary skill in the art is able to make such a determination. Furthermore, if guidance is required in selecting an expression system, one can consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

Eukaryotic cells that contain a nucleic acid molecule that encodes the agent of the invention and express the protein encoded in such nucleic acid molecule in vivo are also features of the invention.

Furthermore, eukaryotic cells of the invention can be cells that are part of a cellular transplant, a tissue or organ transplant. Such transplants can comprise either primary cells taken from a donor organism or cells that were cultured, modified and/or selected in vitro before transplantation to a recipient organism (e.g., eukaryotic cells lines, including stem cells or progenitor cells). Since, after transplantation into a recipient organism, cellular proliferation may occur, the progeny of such a cell are also considered within the scope of the invention. A cell, being part of a cellular, tissue or organ transplant, can be transfected with a nucleic acid encoding a polypeptide of interest and subsequently be transplanted into the recipient organism, where expression of the polypeptide occurs. Furthermore, such a cell can contain one or more additional nucleic acid constructs allowing for application of selection procedures, e.g. of specific cell lineages or cell types prior to transplantation into a recipient organism.

The expressed polypeptides can be purified from the expression system using routine biochemical procedures, and can be used as diagnostic tools or as therapeutic agents, as described below.

Patients Amenable to Treatment: The compositions of the invention are useful in inhibiting T cells that are involved, or would be involved, in an immune response (e.g., a cellular immune response) to an antigen; in inhibiting other cells involved in the pathogenesis of immunological disorders (e.g., monocytes, macrophages, and other antigen presenting cells such as dendritic cells, NK cells, and granulocytes); and in destroying cells such as islet cells (as seen in diabetes), or hyperproliferating cells (as seen, for example, in tissues involved in immunological disorders such as synovial fibroblasts (which are affected in rheumatoid arthritis) keratinocytes (which are affected in psoriasis), or dermal fibroblasts (which are affected in systemic lupus erythematosus). Given these examples, other cell types that can usefully be targeted will be apparent to those of ordinary skill in the art.

Thus, the compositions of the invention can be used to treat patients who are suffering from, or at risk for, an immune disease, particularly autoimmune disease. Examples of autoimmune diseases suitable for treatment are alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

Inflammatory conditions (which are often, but not always, associated with autoimmunity) which may be amenable to treatment are asthma, encephalitis, inflammatory bowel disease, chronic obstructive pulmonary disease (COPD), allergic disorders, pulmonary fibrosis, undifferentitated spondyloarthropathy, undifferentiated arthropathy, arthritis, inflammatory osteolysis, chronic inflammation resulting from chronic viral or bacteria infections, psoriasis (e.g., plaque psoriasis, pustular psoriasis, erythrodermic psoriasis, guttate psoriasis or inverse psoriasis).

Similarly, methods by which these agents are administered can be used to treat a patient who has received a transplant of synthetic or biological material, or a combination of both. Such transplants can be organ, tissue or cell transplants, or synthetic grafts seeded with cells, for example, synthetic vascular grafts seeded with vascular cells. In addition, patients suffering from GVHD or patients who have received a vascular injury would benefit from this method.

In particular, the compositions can be used to treat patients at risk for, or diagnosed with, type I diabetes. The compositions are also useful for treating patients at risk for, or suffering from, type II diabetes.

The invention encompasses administration of target-cell depleting forms of an agent that targets tissue destructive T cells, or inflammatory cells. With target-cell depleting forms of agents, it is possible to selectively kill autoreactive or “transplant destructive” immune cells without massive destruction of other subsets of T cells (e.g., regulatory T cells). Accordingly, the invention features a method of killing cells (e.g., autoreactive Th17 cells, or proinflammatory effector cells such as macrophages). These methods can be carried out by administering to a patient a combination of agents that includes an agent that activates the complement system, lyses cells by the ADCC mechanism, or otherwise kills cells expressing a selected target molecule.

Formulations for Use and Routes of Administration: Although agents of the present invention can be obtained from naturally occurring sources, they can also be synthesized or otherwise manufactured. Polypeptides that are derived from eukaryotic organisms or synthesized in E. coli, or other prokaryotes, and polypeptides that are chemically synthesized will be substantially free from their naturally associated components. In the event the polypeptide is a chimera, it can be encoded by a hybrid nucleic acid molecule containing one sequence that encodes all or part of the agent. Agents of the invention (e.g., polypeptides) can be fused to a hexa-histidine tag to facilitate purification of bacterially expressed protein, or to a hemagglutinin tag to facilitate purification of protein expressed in eukaryotic cells. Where polypeptides are recombinantly produced, codons can be optimized based on the codon preference of the host cell.

In therapeutic applications, agents of the invention can be administered with a physiologically acceptable carrier, such as physiological saline. The therapeutic compositions of the invention can also contain a carrier or excipient, many of which are known to one of ordinary skill in the art. Excipients that can be used include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol. The agents of the invention can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.”

Routes of administration are also well known to skilled pharmacologists and physicians and include intraperitoneal, intramuscular, subcutaneous, and intravenous administration. Additional routes include intracranial (e.g., intracisternal or intraventricular), intraorbital, opthalmic, intracapsular, intraspinal, intraperitoneal, transmucosal, topical, subcutaneous, and oral administration. It is expected that the intravenous or intra-arterial routes will be preferred for the administration of polypeptide agents. The subcutaneous route may also be used frequently as the subcutaneous tissue provides a stable environment for polypeptides, from which they can be slowly released.

In case of cell-based therapies (gene therapies), the cells/tissues/organs could either be transfected by incubation, infusion or perfusion prior to transplantation with a nucleic acid composition, such that the therapeutic protein is expressed and subsequently released by the transplanted cells/tissues/organs within the recipient organism. As well, the cells/tissues/organs could undergo a pretreatment by perfusion or simple incubation with the therapeutic protein prior to transplantation in order to eliminate transplant-associated immune cells adherent to the donor cells/tissues/organs (although this is only a side aspect, which will probably not be of any clinical relevance). In the case of cell transplants, the cells may be administered either by an implantation procedure or with a catheter-mediated injection procedure through the blood vessel wall. In some cases, the cells may be administered by release into the vasculature, from which the subsequently are distributed by the blood stream and/or migrate into the surrounding tissue (this is done in islet cells transplantation, where the islet cells are released into the portal vein and subsequently migrate into liver tissue).

It is well known in the medical arts that dosages for any one patient depend on many factors, including the general health, sex, weight, body surface area, and age of the patient, as well as the particular compound to be administered, the time and route of administration, and other drugs being administered concurrently. Dosages for the polypeptide of the invention will vary, but can, when administered intravenously, be given in doses on the order of magnitude of 1 microgram to 10 mg/kg body weight or on the order of magnitude of 0.01 mg/l to 100 mg/l of blood volume. A dosage can be administered one or more times per day, if necessary, and treatment can be continued for prolonged periods of time. Determining the correct dosage for a given application is well within the abilities of one of ordinary skill in the art.

### Examples

### Example 1

We studied the effects of a therapeutic regimen in the non-obese diabetic (NOD) mouse model of T cell dependent new onset (Shoda et al., Immunity, 23:115-126, 2005) This regimen utilized these three agents: (i) an agonist (wild type) IL-2/Fc fusion protein; (ii) a high affinity IL-15Rα antagonist, mutant IL-15/Fc fusion protein (mIL-15/Fc); and (iii) rapamycin (RPM) (Kim et al., J. Immunol. 160:5742-5748, 1998; Ferrari-Lacraz et al., J. Immunol. 167:3478-3485, 2001). We refer to this combination below as the “three-agent” regimen or as “Power Mix”. The IL-2/Fc fusion protein was used as a component to enhance activation induced cell death (AICD) of effector, but not regulatory, T cells (Zheng et al., Adv. Exp. Med. Biol., 520:87-95, 2003; Li, et al., Nature Med., 7:114-118, 2001). It was also used to provide an IL-2 mediated “non-redundant function in the differentiation of (Foxp3+) regulatory T cells” (Fontenot et al., Nat Immunol., 6:1142-1151, 2005). The mIL-15/Fc fusion blocks proliferation and promotes passive cell death (PCD) of activated effector T cells by aborting proliferative and anti-apoptotic IL-15 signals (Zheng et al., Immunity, 19:503-514, 2003; Li, et al., Nature Med., 7:114-118, 2001; Waldmann, et al., Immunity, 14:105-110, 2001). It also blocks the ability of IL-15 to induce expression of pro-inflammatory cytokines by activated mononuclear inflammatory cells (Zheng, et al., Adv. Exp. Med. Biol., 520:87-95, 2003). RPM blunts the proliferative response of activated T cells to T cell growth factors (TCGF) without inhibiting the AICD signal imparted by IL-2 (Li et al., Nature Med., 5:1298-1302, 1999) or IL-2/Fc.

Moreover, the agonist IL-2 and antagonist IL-15 agents were designed as IgG2a derived Fc fusion proteins to ensure a prolonged circulating half-life and provide a potential means to kill activated effector, but not regulatory, IL-2R⁺ and IL-15R⁺ target cells via the activation of complement and FcR⁺ leukocytes (Zheng et al., Adv. Exp. Med. Biol., 520:87-95, 2003). Hence, the IgG2a-based complement dependent and antibody dependent cell cytotoxicity activating Ig related fusion proteins are potentially cytotoxic proteins that primarily target certain vulnerable activated IL-2R⁺/IL-15R+, not IL-2R⁻/IL-15R⁻ resting, mononuclear leukocytes (Kim et al., J. Immunol., 160:5742-5748, 1998; Zheng et al., J. Immunol., 163:4041-4048, 1999).

We found that treatment with this regimen provided for enduring drug free remission from overt diabetes through ablation of insulitis, restoration of immune tolerance to beta cells, and the unforeseen relief from an inflammatory state in insulin responsive tissues that impairs the ability of these tissues to respond to insulin. We reason that the abolition of beta cell destructive autoimmunity and restoration of self tolerance to insulin producing beta cells is necessary but insufficient to provide long lasting remissions in NOD mice, or perhaps human subjects, with new onset T1DM. The restoration of euglycemia may require ablation of an inflammation imposed insulin resistant state as well as halting the destructive insulitis and restoring immune tolerance to beta cells. Hence, the paucity of success reported to date in creating enduring drug free remissions of T1DM in NODs with narrowly targeted, T cell directed therapies maybe related to an unattended and unforeseen need to ablate inflammation induced insulin resistance. The regimen we used possesses both immune tolerizing and select anti-inflammatory activities, and serves as a prototype for regimens that are well suited for use in restoring euglycemia, particularly in individuals with a modest residual beta cell mass and new onset T1DM.

Short-Term Treatment with Power Mix Restores an Enduring Euglycemic State in Recent Onset Diabetic NOD Mice.

We tested the efficacy of a 14- or 28-day course of Power Mix (RPM+IL-2/Fc+mIL-15/Fc) in new onset (>10 days) T1DM NOD mice whose repeated blood glucose levels ranged from 300 to 450 mg/dl. All non-treated diabetic NOD mice remained hyperglycemic without spontaneous remissions (Table 1, group A) and most died within 7 weeks despite insulin treatment (data not shown). In contrast, euglycemia was achieved within 5-7 weeks and maintained throughout a follow up period of over 300 days in 55 of 60 diabetic NOD mice treated with the RPM+IL-2/Fc+mIL-15/Fc regimen (Table 1 groups B and C). By comparison, remissions were less frequent (Table 1, Groups D-F) and delayed if elements of the treatment mix were eliminated.

To determine whether the presence of the T cell regulatory-enriched CD25+ T cell population is crucial to the beneficial therapeutic effects of Power Mix, prior to administration, we treated new onset diabetic NOD mice with an anti-CD25 mAb regimen known to delete CD4+CD25+ T cells (Zheng et al., Adv. Exp. Med. Biol., 520:87-95, 2003; Sanchez-Fueyo et al., Nat. Immunol., 4:1093-1101, 2003). Seven days after the initiation of anti-CD25 mAb treatment, these CD25+ T cell depleted mice received regimen treatment for 28 days (Table 1, Group G). As a control for the delay in instituting that was imposed by the anti-CD25 pre-treatment regimen, we delayed treatment for 7 days in four NOD mice with new onset diabetes (Table 1, Group H). Each diabetic NOD mouse in the group started on the Power Mix regimen on day 7 without prior administration of anti-CD25 was rendered normoglycemic by day 56 (mean blood glucose level of 129 mg/dl) and remained euglycemic for >200 days of follow up (Table 1, Group H). In contrast, none of the diabetic NOD mice receiving Power Mix after anti-CD25 administration were rendered euglycemic (mean blood glucose level 335 mg/dl) by day 200 (Table 1, Group G). The full beneficial effects of the regimen require the presence of regulatory rich CD25+ cell population. Note that administration of anti-CD25 mAb, but not IL-2/Fc, destroys CD4+CD25+ regulatory T cells (Zheng et al., Immunity, 19:503-514, 2003).

TABLE 1
Short-term treatment Of T1DM NOD mice with IL-2/Fc + mIL-
15/Fc + RPM permanently restores euglycemia.

| | | | | Restored to |
|---|---|---|---|---|
| | | | Duration of | Normoglycemia |
| Group | Treated host | Treatment | Treatment | (#/total treated) |
| A | NOD-sp* | None | None | 0/150 |
| B | NOD-sp* | IL-2/Fc + mIL-15/Fc + RPM | 28 days | 37/40 |
| C | NOD-sp* | IL-2/Fc + mIL-15/Fc + RPM | 14 days | 18/20 |
| D | NOD-sp* | mIL-15/Fc + RPM | 28 days | 9/19 |
| E | NOD-sp* | IL-2/Fc + RPM | 28 days | 10/20 |
| F | NOD-sp* | RPM | 28 days | 5/19 |
| G | NOD-sp*; | mIL-15/Fc + IL-2/Fc + RPM | 28 days | 0/4 |
| | CD25+ depleted | | | |
| H | NOD-sp*; | mIL-15/Fc + IL-2/Fc + RPM | 28 days | 4/4 |
| | non-CD25+ deplete | | | |
| | controls | | | |

*NOD-sp: spontaneous new onset diabetic NOD mice.

Power Mix blocks Autoimmunity and Induces Specific Immune Tolerance to Beta Cells in NOD Mice with New Onset T1DM.

The capacity of Power Mix to destroy or inactivate diabetogenic T cells and/or tilt the balance of anti-islet immunity to toward tolerance was affirmed through experiments in which syngeneic islets were placed into new onset diabetic hosts that are given Power Mix and thus rendered euglycemic. As shown in Table 2, untreated new onset T1DM NOD recipients of syngeneic islets lost graft function and became diabetic at times ranging from 4-21 days post-transplantation (Table 2, Group A) while treatment with a 28-day course of Power Mix started on the day of transplantation enabled permanent acceptance of syngeneic islet grafts (Table 2, Group B).

To determine whether euglycemic, NOD mice treated with Power Mix were rendered tolerant to their islets, we chemically destroyed their beta cells through administration of streptozotocin (stz), a beta cell toxin, long-following (230-330 days) cessation of Power Mix therapy (Table 2, Groups C and D). Subsequently syngeneic islet grafts were transplanted into these successfully treated NOD mice whose diabetic state was rekindled with stz administration. Without re-institution of immunosuppressive therapy, all stz treated recipients of syngeneic islets became normoglycemic within 24 hours and remained normoglycemic thereafter (Table 2, Group C). In contrast to the ready acceptance of syngeneic islet transplants, allogeneic islets are uniformly rejected within 4 weeks of transplantation (Table 2, Group D). Hence, Power Mix created a specific, drug free tolerant state to syngeneic insulin producing beta cells.

TABLE 2
Short-term treatment Of T1DM NOD mice with IL-2/Fc + mIL-15/Fc +
RPM specifically restores immune tolerance to self-beta cells.

| Group | Donor | Recipient | Treatment | Graft Survival (days) |
|---|---|---|---|---|
| A | NOD.SCID | NOD-sp* | no | 4, 7, 8, 10, 12, 21 |
| B | NOD.SCID | NOD-sp* | IL-2/Fc + mIL-15/ | >150**, >200 × 4 |
| | | | Fc + RPM | |
| C | NOD.SCID | NOD-sp/stz*** | no | >100 × 5 |
| D | C57BL/6 | NOD-sp/stz*** | no | 29, 29, 30, 30, 34 |

Syngeneic NOD.SCID islet isografts were transplanted into NOD recipients.
*NOD-sp spontaneous new onset diabetic NOD mice;
**Islet graft removed at >150 day;
***NOD-sp/stz a (streptozotocin) induced diabetic NOD state was induced in NOD recipients. These recipients were previously restored to a euglycemic after onset of diabetes by IL-2/Fc + mIL-15/Fc + RPM therapy. These mice remained euglycemic 230-330 days following the cessation of treatment.

Expression of Cytotoxic T Lymphocyte (CTL)-, Th1- and Pro-Inflammatory Cytokine-Genes within the Pancreatic Lymph Node were Grossly Reduced in Treated Hosts.

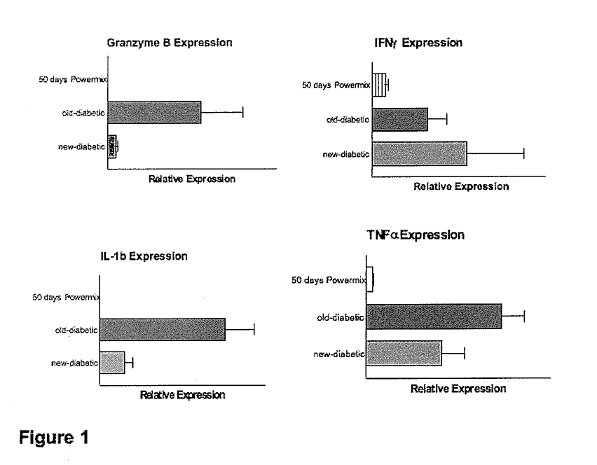

Expression of the CTL-type granzyme B, Th1-type IFNγ and the pro-inflammatory IL-1β and TNFα cytokine genes were grossly reduced in pancreatic lymph nodes in new onset T1DM NOD mice 50-days after initiation of Power Mix treatment as compared to untreated controls (FIG. 1). These data indicate a beneficial inhibitory effect of Power Mix therapy upon local inflammation and upon islet directed cytopathic Th1- and CTL-type immunity. As pro-inflammatory cytokines can exert detrimental effects upon beta cells (Hotamisligil, Nature, 444:860-867, 2006; Eizirik et al., Diabetologia 44:2115-2133, 2001; Sandler et al., Endocrinology, 121:1424-1431, 1987), the marked inhibition of TNFα and IL-1β gene expression in Power Mix treated NOD mice was of considerable interest.

Islet Histology, Beta Cell Mass and Circulating Insulin Levels.

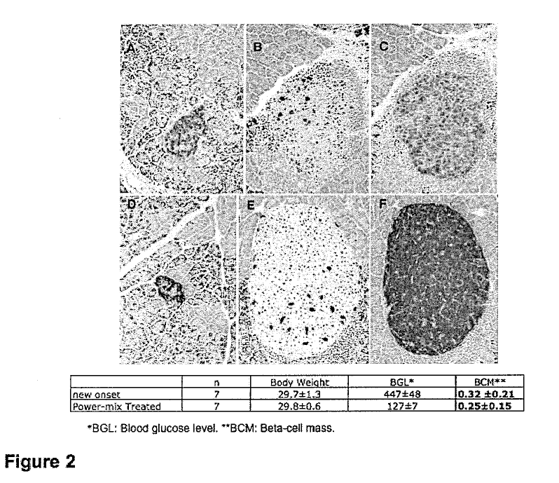

Histologic analysis of islets from spontaneous diabetic NOD mice at the onset of diabetes indicates that (i) most islets are atrophic with few beta cells remaining (unstained central cells in FIG. 2A and FIG. 2B (ii) a minority of islets retain a near normal proportion of beta cells; (iii) leukocytes invade the islets (invasive insulitis); and (iv) the beta cells are partially degranulated (FIG. 2C). In contrast, islet histology of diabetic NOD mice rendered euglycemic by treatment analyzed at least 70 days following cessation of treatment (FIG. 2D-2F) indicated that atrophic islets still are far more common than normal islets (FIG. 2D). Nevertheless, some restoration of the integrity of the remaining islets is manifest in that the residual islets with significant number of beta cells are surrounded, but no longer invaded, by lymphocytes and a higher proportion of beta cells are granulated.

Despite signs of some evidence of improvement among the residual islets, the morphometric analysis revealed an equivalent beta cell mass in recent onset TIDM (n=7, beta cell mass=0.32±0.21 mg) and in three-agent-treated normoglycemic mice (n=7; beta cell mass=0.25±0.15 mg) 70 days or more after onset of TIDM (see Table at the bottom of FIG. 1). For comparison, NOD.SCID mice of 13 and 18 wks of age had a beta cell mass of 1.36±0.12 mg, n=26 (Sreenan et al., Diabetes, 48:989-996, 1999). Thus, both the recent onset and the successfully treated normoglycemic NOD mice bear only 25% of the normal beta cell mass. In short, Power Mix treatment arrests the loss of beta cells, but even the successfully treated mice have no increase in beta cell mass. In humans and in some rodent models a reduction of the beta cell mass to 50% results in diabetes, so there must be heretofore unaccounted factors involved in the return to normoglycemia in treated hosts bearing only 25% of the normal beta cell mass (Weir et al., Diabetes, 53 Suppl 3:S16-21, 2004). To address the possibility that beta cell function improved following treatment and the resulting abatement of pro-inflammatory cytokine expression, we analyzed circulating insulin levels in successfully treated T1DM NODs. Despite the restoration of euglycemia, circulating insulin levels did not rise in successfully treated NODs (data not shown).

Power Mix Treatment Ablates Insulin Resistance in Diabetic NOD Mice.

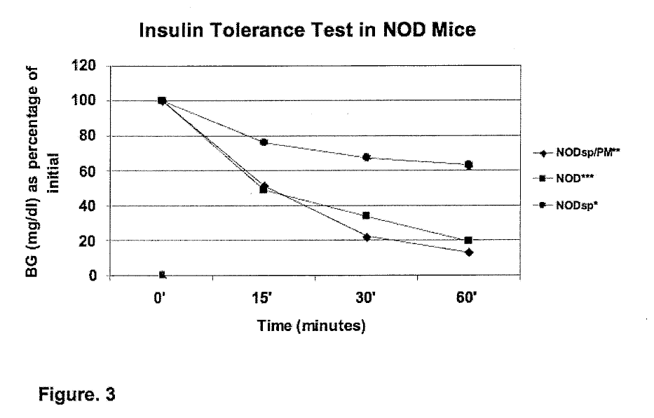

Since hosts successfully treated with Power Mix do not evidence an increase in circulating insulin or a net increase beta cell mass, we sought to determine via insulin tolerance tests whether treatment influences the sensitivity of NOD mice to insulin driven disposal of glucose. Blood glucose levels in 10 week old new onset diabetic mice fell by only 37% over a 1 hr period following an intraperitoneal injection of insulin, but dropped by 81-87% in (i) Power Mix treated and (ii) age matched control non-diabetic NOD mice (FIG. 3). These results demonstrate that the treatment ablates insulin resistance, thereby normalizing the response of host tissues to insulin.

Power Mix Treatment Restores in Vivo Insulin Signaling in Diabetic NOD Mice.

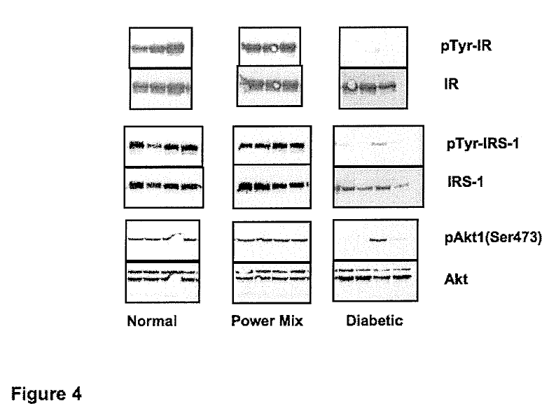

We examined the effects of Power Mix upon insulin signaling in skeletal muscle of new onset diabetic NOD mice in vivo (Shi et al., Diabetes, 55:699-707, 2006). Mice were fasted overnight and injected with human insulin (20 units/kg body weight i.p.) to acutely stimulate insulin signaling. In vivo insulin signaling was monitored by western blot analysis of muscle protein extracts using antibodies specific (i) to tyrosine-phosphorylated insulin receptor (IR), (ii) tyrosine-phosphorylated insulin response substrate-1 (IRS-1) and (iii) PKB/Akt proteins (FIG. 4). Insulin-stimulated tyrosine phosphorylation of IR was markedly diminished in new onset T1DM NOD mice, with a 90% reduction in blot densitometry, compared to age matched control non-diabetic NOD mice (FIG. 4). Impaired insulin signaling was also evident with respect to insulin-stimulated tyrosine phosphorylation of IRS-1 and PKB/Akt, molecules that normally transmit the downstream signals of the insulin activated IR (FIG. 4). As the treatment completely reversed the impaired tyrosine phosphorylation of IR, IRS-1 and PKB/AKT in new onset T1DM NOD mice, it ablates insulin resistance (FIG. 3) via restoration of insulin signaling (FIG. 4) in NOD mice.

Power Mix Treatment Dampens Expression of Inflammatory Genes.

Using reverse transcriptase assisted polymerase chain reaction (RT-PCR) methodology, a targeted transcriptional profile for select inflammation-associated gene expression events within muscle and fat, key tissues for insulin driven disposal of glucose, was compiled in NOD mice (Table 3). The impact of short term Power Mix therapy upon transcriptional profiles in new onset T1DM mice rendered euglycemic by Power Mix therapy was compared with a transcriptional profile obtained with mice rendered euglycemic from the time of diagnosis of T1DM with intense insulin therapy delivered with osmotic pumps. Power Mix therapy, unlike insulin pump therapy, does not immediately render the treated mice euglycemic. As Power Mix treated mice remain hyperglycemic for 5 to 7 weeks we temporarily used non-intensive, conventional insulin therapy delivered (i.p.) in Power Mix treated hosts to prevent extreme hyperglycemia until the advent of euglycemia (at which time insulin therapy is discontinued). Hence, we also analyzed insulin sensitive tissues by RT-PCR in new onset T1DM mice treated by conventional insulin treatment for 5 to 7 weeks (chronic diabetic group). As compared to both control groups (chronic diabetic and osmotic insulin pump treated NODs), expression of several pro-inflammatory cytokines, acute phase-, and other inflammation associated-genes were markedly decreased in fat (Table 3, n=5 for each data point) and muscle (data not show; n=3 for each data point) of Power Mix treated new onset T1DM NODs (Table 3). Power Mix treatment, as compared to samples obtained from chronic diabetic and normal NOD mice, led to reduced expression of these genes. While osmotic insulin pump therapy as compared to conventional insulin treated chronic diabetic NODs, reduced expression of some inflammation associated genes (e.g., TNFα, SOCS2), the effects were not as broad or as potent as those produced by Power Mix (Table 3). Interestingly, expression of the TGF-β anti-inflammatory gene was not dampened by Power Mix therapy.

TABLE 3
IL-2/Fc + mIL-15/Fc + RPM treatment of diabetic NOD mice reduces
intra-adipose expression of inflammation associated genes.

| Groups Compar [symbol] | SOCS1 | SOCS2 | TNFα | C3 | Cp | CRP | GBP1 | IL-1β | PAI-1 | SAA-1 | TGFβ |
|---|---|---|---|---|---|---|---|---|---|---|---|
| Power Mix vs. | * | NS | NS | ** | ** | NS | ** | NS | ** | ** | * |
| Osmotic Pump | | | | | | | | | | | |
| Power Mix vs. | ** | NS | ** | ** | NS | ** | * | NS | NS | NS | NS |
| Chronic Diabetic | | | | | | | | | | | |
| NOD | | | | | | | | | | | |
| Power Mix vs. | *** | * | ** | *** | * | *** | NS | * | NS | NS | NS |
| Normal NOD | | | | | | | | | | | |

Table 3: RT-PCR results from comparing Power Mix treated NOD mice to different control groups (Mann-Whitney test was used for data analysis).
NS = Not significant,
* = 0.05,
** = 0.01,
*** = 0.001
SOCS1 = Suppressor of cytokine signalingl,
SOCS2 = Suppressor of cytokine signaling2,
TNFα = Tumor necrosis factor α,
C3 = Complement 3,
Cp = Ceruloplasmin,
CRP + C-reactive protein,
GBP = 1 Guanylate nucleotide binding protein-1,
IL-1β = Interleukin-1β,
PAI-1 = Plasminogen activator inhibitor type-1,
SAA-1 = Serum amyloid A-1,
TGF-β = Transforming growth factor-β [symbol]
indicates data missing or illegible when filed

Adoptive Transfer Experiments and Power Mix Therapy.

Splenic leukocytes harvested from insulin treated spontaneously diabetic female NOD mice were adoptively transferred into NOD.SCID mice (Table 4). The adoptive transfer of 100×10⁶ splenic leukocytes from diabetic NOD hosts resulted in rapid onset of diabetes in NOD.SCID cell transfer recipients (n=3) within two weeks (9-13 days, Table 4, Group A). In contrast, a 28-day course of Power Mix treatment protected 100% of NOD.SCID recipients (n=10) from autoimmune diabetes for at least 165 days after transfer of 100×10⁶ splenic leukocytes from diabetic NOD mice (Table 4, Group B).

To determine whether Power Mix failed to eliminate diabetogenic T-cells in the few treated diabetic NOD mice that did not become euglycemic, another cell transfer experiment was performed. Splenic leukocytes (100×10⁶) from Power Mix treated diabetic NOD mice were adoptively transferred into NOD.SCID mice (Table 4, Group C). In comparison to the results obtained in untreated hosts receiving 100×10⁶ spleen cells from untreated new onset diabetics (Table 4, Group A), the onset of diabetes was delayed in hosts that received 100×10⁶ spleen cells from Power Mix treated diabetic (Power Mix failures) donors (Table 4, Group C). Hence, Power Mix therapy does eliminate or inactivate many, not all, diabetogenic T cells in treated NOD mice, even in NOD mice that did not achieve euglycemia.

To further investigate whether administration of the therapy directly targets the diabetogenic effector T cells, CD25− T cells were isolated from splenic leukocytes and adoptively transferred into NOD.SCID mice. Following the adoptive transfer of 55×10⁶ CD25− T cells from untreated, spontaneously diabetic NOD mice, diabetes was noted by 21 days in all 10 NOD.SCID cell transfer recipients (Table 4, Group D). In contrast, none of the Power Mix treated NOD.SCID recipients of 55×10⁶ CD25− T cells became diabetic by 21 days post cell transfer (Table 4, Group E). Indeed 2 of 5 of these recipients have remained euglycemic throughout the follow up period (>110 days; Table 4, Group E). While complete protection from diabetes was noted in Power Mix treated NOD.SCID mice that were recipients of whole splenic leukocyte cell transfers from diabetic NOD mice, the same treatment protected only 2 of 5 recipients from eventual diabetes that received CD25+ depleted T cell populations (Table 4; Groups B vs. E). Taken together the data shown in Table 2 indicates that Power Mix treatment protects from autoimmunity in a CD25+ T cell dependent process via an effect that targets autoimmune effector T cells for inactivation or elimination.

TABLE 4
IL-2/Fc + mIL-15/Fc + RPM blocks the development
of T1DM in a passive transfer model.

| | Donor | | Treatment | | T1DM Onset |
|---|---|---|---|---|---|
| Group | (leukocytes) | Recipient | Donor | Recipient | (days post-adoptive transfer) |
| A** | NOD-sp* | NOD.SCID | no | no | 9, 11, 13 (n = 3) |
| B** | NOD-sp* | NOD.SCID | no | IL-2/Fc + | >165 (n = 10) |
| | | | | mIL-15/Fc + | |
| | | | | RPM | |
| C** | NOD-sp*⁺ | NOD.SCID | IL-2/Fc + mIL- | no | 29, 33, 41, >45 |
| | | | 15/Fc + RPM | | |
| D*** | NOD-sp* | NOD.SCID | no | no | 15, 19, 19, 19, 20, |
| | (CD25−) | | | | 20, 20, 20, 20, 21 |
| E*** | NOD-sp* | NOD.SCID | no | IL-2/Fc + | 30, 43, 50, >110, >125 |
| | (CD25−) | | | mIL-15/Fc + | |
| | | | | RPM | |

*NOD-sp: spontaneous new onset diabetic NOD mice.
**100 × 10⁶ unfractionated spleen cells were transferred;
***55 × 10⁶ CD25⁺ depleted cells were transferred;
***A 28-day course of IL-2/Fc + mIL-15/Fc + RPM was used;
⁺The donors of the cell transfers in Group C were new onset diabetic NOD mice that failed to become euglycemic following IL-2/Fc + mIL-15/Fc + RPM treatment.

In summary, we found that a 14- or 28-day course of Power Mix therapy restored an enduring euglycemic state in 55 out of 60 treated, spontaneously and acutely diabetic NOD mice within 5-7 weeks of initiation of treatment. In parallel, the autoimmune islet destructive T cell rich insulitis process was aborted and a discriminating state of immune tolerance to “self”-islet beta cells was restored. Several other lines of evidence demonstrate that aggressive, beta cell directed autoimmunity was markedly curtailed as a consequence of this treatment. While treatment destroys or inactivates beta cell destructive T cell populations, deletion of the regulatory T cell rich population of CD25+ T cells prior to treatment precludes restoration of a euglycemic state in treated new onset diabetic NOD mice. The importance of preservation of the regulatory T cell rich-CD25+ T cell populations following Power Mix therapy was also evident the NOD passive transfer model (Table 4). Overall, Power Mix induces specific tolerance and tips the immune balance from diabetogenic toward beta cell protective immunity.

Although treatment served to halt the progressive and destructive autoimmune insulitis, morphometric analysis showed, to our surprise, no apparent difference in beta cell mass or in circulating insulin levels between recent onset TIDM and Power Mix treated normoglycemic NOD mice. Both recent onset T1DM and formerly T1DM mice rendered normoglycemic by Power Mix treatment bear only 25% of the normal beta cell mass and circulating insulin levels remained low and unchanged. Clearly, restoration of euglycemia in treated T1DM NOD mice is not created through gross expansion of the beta cell mass or marked improvements in circulating insulin levels. Hence, we sought to determine whether new onset T1DM NOD mice exhibit insulin resistance as well as destruction of insulin producing beta cells.

Infiltration of activated macrophages or expression of pro-inflammatory cytokines and proteins within critical insulin sensitive tissue is known to hamper insulin responsiveness and insulin signaling in obesity linked type II diabetes mellitus (T2DM) (Hotamisligil, Nature, 444:860-867, 2006; Shoelson et al., J. Clin. Inv., 116:1793-1801, 2006). Chaparro et al. recently reported that new onset T1DM NOD mice do indeed manifest an insulin resistant state (Proc. Nat. Acad. Sci. USA, 103:12475-12480, 2006). We confirmed and extended this observation. In the course of our work, we tested the hypothesis that insulin resistance may be linked by expression of pro-inflammatory molecules within fat and muscle that are crucial for insulin triggered disposal of glucose, and that resolution of an inflammation-associated insulin resistant state and of faulty insulin triggered tyrosine phosphorylation of insulin signaling molecules may be linked to restoration of euglycemia.

Indeed, Power Mix treatment serves to ablate insulin resistance and to restore normal tyrosine phosphorylation linked insulin signaling in new onset T1DM NOD mice. A transcriptional profiling approach provided evidence that restoration of euglycemia and ablation of insulin resistance with treatment is associated with a significant reduction in intra-fat/muscle expression of a variety of genes known to be hyper-expressed within inflamed tissues although expression of the anti-inflammatory TGF-β gene was not impacted. It is particularly pertinent that Power Mix therapy induced relief of insulin resistance occurs in concert with a gross reduction of inflammation related gene expression events within fat and muscle as expression of these molecules are known to cause insulin resistance in certain forms of T2DM (Hotamisligil, Nature 444:860-867, 2006). The molecular signature of inflammation impaired insulin signaling in vivo is defective insulin triggered tyrosyl phosphorylation of the insulin receptor (Hotamisligil, Nature 444:860-867, 2006). Inflammatory signals are known to disrupt insulin stimulated tyrosyl phosphorylation of the insulin receptor and other downstream signaling molecules, a necessary action for insulin triggered signal transduction (Bruning et al., Cell 88:561-572, 1997). That Power Mix treatment restored insulin stimulated tyrosyl phosphorylation of the insulin receptor, IRS-1 and other downstream signaling molecules provides a mechanism by which Power Mix therapy may resolve the insulin resistance. Other inflammatory proteins can also impair insulin signals albeit by mechanisms other than faulty insulin triggered tyrosine phosphorylation (Hotamisligil, Nature 444:860-867, 2006; Howard et al., Trends Endocrin. Metab. 17:365-371, 2006). In short, Power Mix therapy grossly dampens the pattern of inflammation associated insulin resistance and faulty tyrosine phosphorylation of critical proteins in the insulin signaling cascade but does not lead to an increase in circulating insulin or the beta cell mass. Therefore, it seems likely that that the relief from insulin resistance is a critical factor in the restoration of euglycemia induced by treatment. Our data indicate that relief of unappreciated inflammation-induced state of insulin resistance and faulty insulin triggered tyrosine phosphorylation events as well as the long recognized requirement for ablation of the beta cell destructive autoimmune insulitis and restoration of self-tolerance to islets are required to permanently restore euglycemia in new onset T1DM hosts. In this respect, it is notable that the treatment regimen includes an IL-15R antagonist and IL-15 is known to trigger the expression of pro-inflammatory cytokines (Ferrari-Lacraz et al., J. Immunol. 173:5818-5826, 2004; Zheng et al., Adv. Exp. Med. Biol. 520:87-95, 2003). Few T cell directed therapies tested to date have proven successful in restoring euglycemia in the new onset NOD model.

Materials and Methods:

Mice: Female NOD (NOD/LtJx) mice and NOD.SCID (NOD.CB17-Prkdc^{scid}/J) were purchased from Jackson Laboratories (Bar Harbor, Me.) at 4 weeks of age and maintained under pathogen-free conditions at the Massachusetts General Hospital (Boston, Mass.). All animal studies were approved by our institutional review board.

Blood glucose levels of NOD mice were monitored twice weekly with the Accu-Check blood glucose monitor system (Roche, Indianapolis, Ind.). When non-fasting blood glucose levels are in excess of 300 mg/dl on two consecutive measurements, a diagnosis of new onset of diabetes is made. For syngeneic islet transplant recipients, blood glucose levels were checked at the time of transplantation, then daily for two weeks, and then 2 to 3 times per week afterward.

Induction of and management of diabetes: Successfully treated euglycemic NOD mice were rendered hyperglycemic with stz (275 mg/kg i.p) treatment 230 to 300 days following the original spontaneous onset of diabetes. With the re-emergence of hyperglycemia following stz administration, these diabetic NOD mice were used as syngeneic or allogeneic islets graft recipients. Graft failure was defined as the first day of 3 consecutive days of blood glucose levels >250 mg/dl.

Islet transplantation: NOD.SCID mice and C57BL/6 mice (10-12 weeks old) were used as donors for islet transplants. Islets were isolated using a modification of the method of Gotoh et al. (Transplantation, 40:437, 1985), in which the pancreatic duct is distended with collagenase P. After Histopaque gradient (Histopaque^{R}-1077, Sigma Chemical Co., St. Louis, Mo.) purification, islets with diameters between 75 and 250 μm were hand picked and transplanted under the renal capsule. Each recipient received 600-800 NOD.SCID or C57BL/6 islets.

Reagents and treatment protocols: The mutant IL-15/Fc and IL-2/Fc proteins used for experiments involving the NOD mice were designed, expressed and purified as previously described (Kim et al., J. Immunol., 160:5742-5748, 1998; Zheng et al., J. Immunol., 163:4041-4048, 1999). A rat anti-mouse CD25 (PC61 5.3, IgG1, ATCC TB222) producing hybridoma was purchased from American Type Culture Collection (Rockville, Md.) and grown in SFM hydridoma media (Invitrogen, Carlsbad, Calif.). The anti-CD25 mAb was purified by protein G affinity chromatography. Rapamycin was purchased from the Massachusetts General Hospital pharmacy.

The Power Mix treatment regimen for mice includes antagonist-type mutant IL-15/Fc, wild type IL-2/Fc proteins and RPM. RPM was given i.p. at a dose of 3 mg/kg daily for the first 7 days, and every other day thereafter for total 14 or 28 days. IL-2/Fc and mIL-15/Fc proteins were administered (5 μg i.p. daily) for 14 or 28 days. In some experiments RPM alone or RPM plus one, but not both, fusion proteins were administered using the aforementioned dosing regimen.

To deplete CD25+ T cells, new onset NOD mice were treated with 3 doses of anti-CD25 mAb (PC 61) at days 7, 5, and 3 prior to initiation of Power Mix treatment (Sanchez-Fueyo et al., Nat. Immunol., 4:1093-1101, 2003).

Insulin tolerance test: Insulin tolerance tests (ITT) (Bruning et al., Cell, 88:561-572, 1997), were performed in age matched NODs including 1) spontaneous new onset diabetic NOD mice (NOD-sp); 2) Power Mix treated spontaneous new onset NOD mice (NOD-sp/PM); 3) non-diabetic NOD mice. Food was withheld 3 hours before testing. Animals were weighed and blood samples collected just before the injecting the animals with 0.75 U/kg of regular human insulin (i.p.) (Novolin, Novo Nordisk Pharmaceutical Industries, Inc. Clayton, N.C.). Blood samples were then collected at 15, 30 and 60 minutes after the insulin injection. The results were expressed as percentage of initial blood glucose concentration (Bruning et al., Cell, 88:561-572, 1997).

Morphometric analysis of beta cell mass: Animals were anesthetized by Nembutal, pancreases were excised, weighed, fixed in Bouin's solution and embedded in paraffin. Islet sections (5 μm) were immunostained (peroxidase-antiperoxidase) using rabbit anti-bovine glucagon (1:3000, gift of Dr. M. Appel) or anti-insulin (1:200, Linco). Beta cell mass was measured by point counting morphometry: one full footprint section of each pancreas was scored systematically at a magnification of 420× using a 90 point grid to obtain the number of intercepts over beta cell, alpha cell, exocrine pancreatic tissue and non-pancreatic tissue; 200-500 fields per animal were counted. The beta cell relative volume (intercepts over beta cells divided by intercepts over total pancreatic tissue) was multiplied by the pancreas weight to calculate the beta cell mass (Xu et al., Diabetes, 48:2270-2274, 1999).

PCR Methods: To quantitatively analyze gene expression profiles, pancreatic draining lymph nodes were harvested from pre-diabetic, newly diabetic (onset of T1DM within one week), old diabetic (diabetic more than 30 days), and Power Mix treated new onset diabetic mice (at day 50 following initiation of treatment). Messenger RNA was extracted using an RNeasy mini-kit (Qiagen). Reverse transcription to cDNA was performed using TaqMan Reverse Transcription reagents obtained from Applied Biosystems (Foster City, Calif.). Specific message levels were quantified by real time PCR using the ABI 7700 Sequence Detection System (Applied Biosystems). Amplification was performed for a total of 40 cycles and target gene products were detected using gene specific primers and FAM labeled probes designed by Applied Biosystems. A GAPDH primer and VIC labeled probe were used as the internal control (Applied Biosystems). Quantification of all target genes was based on a standard comparative threshold cycle (Ct) method.

To quantitatively analyze gene expression profiles, fat (n=for each data point) and muscle (n=3 for each data point) were harvested from pre-diabetic, newly diabetic (onset of T1DM within one week), old diabetic (diabetic more than 30 days), and Power Mix treated new onset diabetic mice (at day 50 following initiation of treatment). Messenger RNA was extracted from fat and muscle using Invitrogen's Micro to Midi kit (Carlsbad, Calif.) according to the manufacturer's protocol. Reverse transcription to cDNA was performed using TaqMan Reverse Transcription reagents obtained from Applied Biosystems (Foster City, Calif.) (Li et al., 2001). Oligonucleotide primers and fluorogenic probes were designed and synthesized and tested for validity for the measurement of mRNA levels of Suppressor of cytokine signaling1 (SOCS1), Suppressor of cytokine signaling2 (SOCS2), tissue necrosis factor a (TNFα), Complement 3 (C3), Ceruloplasmin (Cp), C-reactive protein (CRP), Guanylate nucleotide binding protein-1 (GBP1), interleukin-1β (IL-1β, plasminogen activator inhibitor type-1 (PAI-1), Serum amyloid A-1 (SAA-), transforming growth factor-β (TGF-β). To measure mRNA levels of the internal control GAPDH a commercially available probe and primer mix (Applied Biosystems) were used. PCR analysis was performed by a two-step process. In the first step, a pre-amplification reaction was set up using the ABI bio-systems thermo cycler with 3 μl cDNA and 7 μl of dNTP, 10× PCR buffer, Taq DNA polymerase, and gene specific oligonucleotide primer pairs. This was followed by measurement of mRNA with an ABI Prism 7900HT sequence detection system. PCR reactions for all the samples were set up in duplicates as a 25 μl reaction volume using 12.5 μl TaqMan Universal PCR Master Mix, 2.5 μl pre-amplified template cDNA, 300 nM primers and 200 nM probe. PCR amplification protocol included 40 cycles of denaturing at 95° C. for 15 sec and primer annealing and extension at 60° C. for 1 minute. Transcript levels were calculated using standard curve method (Ding, et al., Transplantation, 75:1307-1312, 2003). The PCR amplicon for 18S rRNA was kindly provided by Dr. Suthanthiran, Weill Medical College of Cornell University, New York, USA. 18S rRNA amplicon was quantified and used for developing standard curves. The standard curves were based on the principle that a plot of the log of the initial target copy number of a standard versus threshold cycle results in a straight line. Messenger RNA levels in the samples were expressed as number of copies per microgram of total RNA isolated from fat and muscle. Messenger RNA copy numbers were normalized with the use of GAPDH copy numbers (the number of mRNA copies in 1 μg of RNA divided by the number of GAPDH mRNA copies in 1 ug of RNA). In the absence of detectable level of a transcript, a value equal to half the minimum observed GAPDH-normalized level was assigned (Helsel, Environ Sci Technol, 24:1766-1774, 1990).

In vivo insulin signaling studies: In vivo insulin signaling experiments were performed on mice after a 16 hr fast. Mice were injected i.p. with 20 U/kg of human insulin (Eli Lilly) or saline. Fat and skeletal muscle (gastronemius) were dissected and frozen in liquid nitrogen for immunoblotting analysis of insulin signaling proteins.

Immunoblotting: Fat and skeletal muscle (gastronemius) from the in vivo insulin signaling studies were homogenized in a modified radioimmunoprecipitation assay (RIPA) buffer containing 50 mM Tris-HCl, 1 mM EDTA, 1% NP-40, 0.25% sodium deoxycholate, 150 mM NaCl, 1 mM PMSF, 200 μM Na₃VO₃, supplemented with 1% protease inhibitor cocktail (Sigma), and 1% tyrosine phosphatase inhibitor cocktail (Sigma). Cell homogenates were incubated on ice for 45 min to solubilize all proteins, and insoluble portions were removed by centrifugation at 14,000 g at 4° C. for 15 min. Whole cell lysates were separated by SDS-polyacrylamide gel electrophoresis (PAGE). Proteins on the gels were transferred to Hybond ECL nitrocellulose membrane (Amersham Pharmacia Biotech, Piscataway, N.J.). The transferred membranes were blocked, washed, incubated with various primary antibodies, and followed by incubation with horseradish peroxidase-conjugated secondary antibodies. Rabbit polyclonal anti-IR (pY1162/1163) and anti-IRS-1 (pY612) antibodies were purchased from BioSource (BioSource International, Inc., Camarillo, Calif.). Rabbit polyclonal anti-IR antibody was purchased from Santa Cruz Biotech (Santa Cruz, Calif.). Rabbit polyclonal anti-IRS-1 was obtained from Upstate (Lake Placid, N.Y.). Visualization was done with a chemiluminescence reagent, using the ECL Western Blotting Analysis System (Amersham Pharmacia Biotech). The blots were quantified using densitometry (Molecular Dynamics, Sunnyvale, Calif.).

Adoptive transfer studies: First, 100×10⁶ spleen cells harvested from chronically diabetic NOD mice were adoptively transferred into NOD.SCID mice. The NOD.SCID mice were randomly divided into treatment and control groups. The NOD.SCID mice in treatment group received Power Mix treatment for 28 days starting on day −2 related to the time of adoptive cell transfer. Power Mix treatment was administered using the aforementioned dosing regimen. In a second set of experiments, we transferred spleen cells from control or Power Mix treated diabetic NOD mice. Unfractionated splenic mononuclear leukocytes (55×10⁶) as well as purified CD25− T cells were prepared as previously described (Sanchez-Fueyo et al., J. Immunol., 168:2274-2281, 2002) and used in passive transfer experiments.

### Example 2

In this example, we demonstrate that treatment with α1 antitrypsin (AAT), an agent dampens inflammation but does not directly inhibit T cell activation, ablates invasive insulitis and restores euglycemia, immune tolerance to beta cells, normal insulin signaling and insulin responsiveness in NOD mice with recent onset T1DM. Indeed, the mass of insulin producing beta cells expands in AAT treated diabetic NOD mice.

AAT Does Not Inhibit T Cell Activation.

To test the hypothesis that human AAT does not directly act upon T cells, carboxyfluorescein diacetate succinmidyl ester (CFSE)-labeled C57BL/6 mouse T cells were stimulated with plate bound anti-CD3 plus soluble anti-CD28 mAbs. AAT did not inhibit proliferation or the cell surface expression of CD25, CD62L, and CD44 T cell activation proteins. The data are in accord with the failure of AAT to bind to T cells (Arora et al., Nature, 274:589-590, 1978), or inhibit Con A induced T cell proliferation. (Lewis et al., Nat. Immunol., 2007).

Short-Term AAT Treatment Restores an Enduring Euglycemic State in New Onset Diabetic NOD Mice.

We tested the efficacy of a short (2 mg i.p. every 3 days×5) course of human AAT in new onset (>10 days) T1DM NOD mice whose thrice repeated blood glucose levels ranged from 300 to 450 mg/dl. All untreated diabetic NOD mice remained hyperglycemic without spontaneous remissions (Table 5, group A) and most died within 7 weeks despite insulin treatment (data not shown). In contrast, euglycemia was achieved in 14 of 16 AAT treated diabetic NOD mice, 12 achieving euglycemia within 3 weeks, and euglycemia was maintained indefinitely (throughout a follow up period of over 270 days) despite cessation of therapy (Table 5, Group B). Thus, human AAT, an acute phase reactant protein, with powerful anti-inflammatory properties, (Lu et al., Hum. Gene Ther., 17:625-634, 2006; Churg et al., Lab. Inv., 81:1119-1131, 2001; Jie et al., Chinese Med. J., 116:1678-1682, 2003; Lewis et al., Proc. Natl. Acad. USA, 102:12153-12158, 2005; Petrache et al., Am. J. Resp. Crit. Care Med., 173:1222-1228, 2006; Lewis et al., Nat. Immunol., 2007), but lacking direct effects upon T cells, (Arora et al., Nature, 274:589-590, 1978), is an exceptionally potent therapy for the treatment of new onset T1DM in the NOD model. These data are consistent with the hypothesis that inflammation triggers new onset T1DM.

TABLE 5
Short-term AAT treatment of NOD mice restores euglycemia.

| | | Normoglycemia | Normoglycemic/ | |
|---|---|---|---|---|
| Groups | | achieved | Total number | Days after |
| (n) | Treatment | (range in days) | of mice used | treatment |
| A | None | Never | 0/150 | 50 |
| n = 150 | | | | |
| B | AAT | 1-22 (49, 55)* | 14/16 | 150-270 |
| n = 16 | | | | |

*12 mice treated with AAT became normoglycemic within 22 days after initiation of therapy. The other two mice that became normoglycemic at 49 and at 55 days.

Islet Histology, Beta Cell Mass and Circulating Insulin Levels.

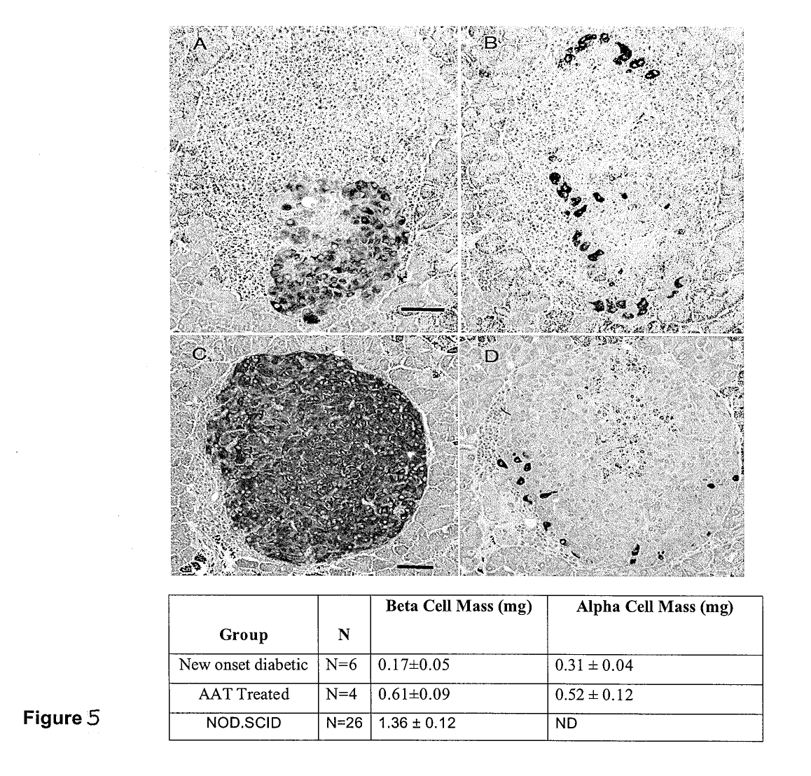

Histologic analysis of islets obtained from spontaneously diabetic NOD mice at the onset of overt hyperglycemia indicate that (i) most islets are atrophic with few beta cells remaining (data not shown) (ii) a minority of islets retain a substantial number of beta cells and normal numbers of alpha cells (FIG. 5A, 5B); (iii) leukocytes invade the islets (invasive insulitis) (FIG. 5A, 5B); and (iv) the beta cells are partially degranulated (FIG. 5A). In contrast, islet histology of diabetic NOD mice rendered euglycemic by human AAT treatment analyzed at least 35 days following cessation of AAT treatment (FIG. 5C, 5D) show regranulation of the beta cells and a greater proportion of beta to alpha cells. Small atrophic islets have slightly larger islands of beta cells than at onset. The large beta cell-rich islets are surrounded, but not invaded, by lymphocytes (FIG. 5C, 5D). Islets now manifest distinct smooth edges, a pattern consistent with eradication of invasive insulitis (FIG. 5C, 5D). The change from invasive to circumferential insulitis has been linked with the induction of tolerance to islets. (Rossini et al., Annu. Rev. Immunol., 3:289-320, 1985; Shoda et al., Immunity, 23:115-126, 2005).

The beta cell mass (BCM) was significantly increased as compared to the BCM of new onset diabetic NOD mice (2 tailed unpaired t test, p=0.004). An apparent regeneration of the beta cells was discerned. For comparison, non-diabetes prone NOD.SCID mice at 13 and 18 wks of age had a BCM of 1.36±0.12 mg, n=26 (Sreenan et al., Diabetes, 48:989-996, 1999). (Table at the bottom of FIG. 5). The BCM of recent onset diabetic NODs was only ca. 10% of that of adult NOD-SCID mice, while the BCM of mice who recovered euglycemia following AAT treatment quickly rose to 50% of the normal BCM for NOD-SCID mice (Table at the bottom of FIG. 5). Interestingly, the mass of glucagon positive alpha cells did not rise as a consequence of AAT treatment. The presence of slightly larger islands of non glucagon positive cells in many atrophic islets and the massively increased beta cell to alpha cell ratio in the few large beta cell-rich residual islets in these AAT treated NODs suggest beta cell regeneration, perhaps from residual beta cells, was fostered by AAT treatment. In short, AAT treated animals had a significantly increased beta cell mass as compared to untreated controls. To further address the possibility that beta cell function improved following AAT treatment, we analyzed circulating fasting insulin levels in successfully treated T1DM NOD mice. Indeed, circulating fasting insulin levels did rise in euglycemic AAT treated NOD mice compared to the newly diagnosed diabetic NOD mice (Table 6).

TABLE 6
Circulating fasting insulin levels in
successfully treated T1DM NOD mice.

| | | Insulin | | Insulin |
|---|---|---|---|---|
| Animal # | (Day)* | (ng/ml) | (Day)** | (ng/ml) |
| 1 | 0 | 1.051 | 6 | 1.606 |
| 2 | 0 | 0.549 | 6 | 0.835 |
| 3 | 0 | 1.18 | 7 | 1.654 |
| 1 | 0 | 1.508 | 15 | 2.128 |
| 2 | 0 | 1.051 | 15 | 1.606 |

*Day 0 at which time AAT treatment commenced in new onset T1DM NOD mice.
**The number of days after initiation of therapy at which time restoration of normoglycemia was evident.

AAT Treatment Aborts Diabetogenic Autoimmunity and Induces Specific Immune Tolerance to Beta Cells in NOD Mice with New Onset T1DM.

Despite the lack of a direct effect upon T cells, the capacity of AAT treatment to tilt the overall balance of anti-islet immunity away from islet cell destructive immunity and toward tolerance was affirmed through experiments in which syngeneic islets were placed into new onset diabetic hosts that had been successfully treated with AAT and thereby now rendered euglycemic. As shown in Table 7, control untreated new onset T1DM NOD recipients reject syngeneic islet grafts and become diabetic 4-21 days post-transplantation (Table 7, Group A). To determine whether euglycemic AAT treated NOD mice were rendered tolerant to their islets, we chemically destroyed their remnant beta cells through administration of streptozotocin (stz), a beta cell toxin, long-following (200-300 days) cessation of AAT (Table 7, Groups B, C). Subsequently syngeneic islet grafts were transplanted into successfully treated NOD mice whose diabetic state was rekindled with stz administration (Table 7, Groups B). Without re-institution of immunosuppressive therapy in hosts previously treated with AAT, all stz treated recipients of syngeneic islets became normoglycemic within 24 hours and remained normoglycemic thereafter (Table 7, Group B). In contrast to the ready acceptance of syngeneic islet transplants in NOD recipients who had been previously (ca. 200-300 days) rendered euglycemic by AAT treatment, allogeneic islets are uniformly rejected within 11 days of transplantation (Table 7, Group C). Despite the absence of known direct effects of AAT upon T cells, AAT treatment creates a specific, drug free tolerant state to syngeneic insulin producing beta cells. Of course, allogeneic islets transplanted into spontaneously diabetic NOD mice treated with stz are rapidly rejected (data not shown).

TABLE 7
Short-term treatment of T1DM NOD mice with AAT specifically
restores immune tolerance to beta cells.

| | | | Prior | Graft Survival |
|---|---|---|---|---|
| Group | Donor | Recipient | treatment | (days) |
| A | NOD.SCID | NOD-sp* | NONE | 4, 7, 8, 10, 12, 21 |
| B | NOD.SCID | NOD-sp/stz** | AAT | >53, >54, >60, >61 |
| | | | | >71, >81 |
| C | C57BL/6 | NOD-sp/stz** | AAT | 4, 7, 9, 11, |

*NOD-sp spontaneous new onset diabetic NOD mice;
**NOD-sp/stz a streptozotocin induced diabetic state was induced in NOD recipients. Spontaneously diabetic NOD mice were previously restored to a euglycemic after onset of diabetes by AAT therapy. These mice remained (Group B, C) euglycemic 200-300 days following the cessation of treatment. Syngeneic (Group A, B) NOD.SCID islet or allogeneic C57BL/6 (Group C) islet grafts were transplanted into NOD recipients.

AAT Treatment Alters the Balance of Immunity and Inflammation in the Pancreatic Lymph Node.

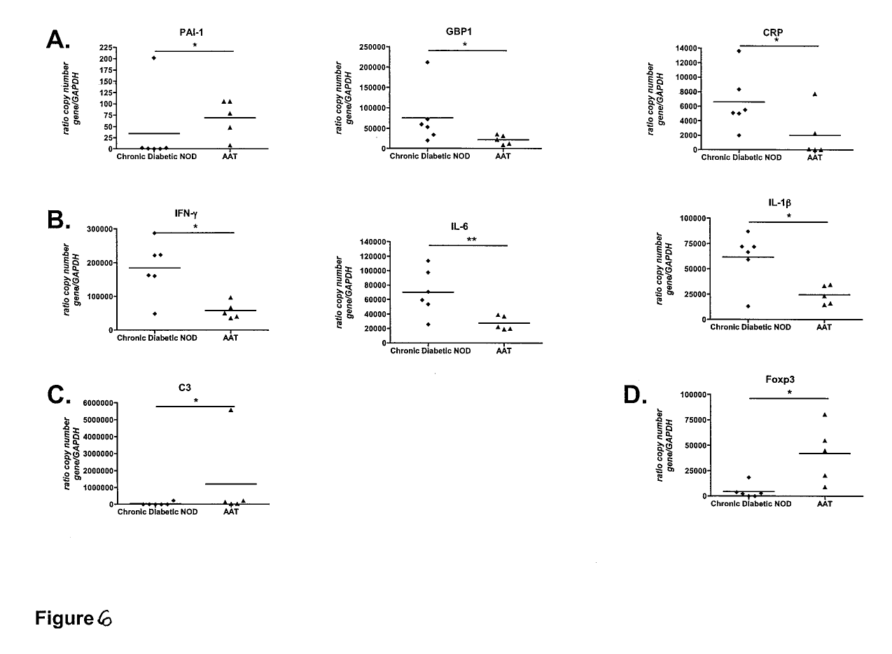

To further analyze the impact of AAT treatment on beta cell directed autoimmunity, a targeted reverse transcriptase assisted polymerase chain reaction (RT-PCR) approach was applied. In this analysis we compared transcriptional profiles of pancreatic lymph node samples obtained from mice that were rendered euglycemic by AAT treatment with samples obtained from new onset diabetic NOD mice that were treated with insulin (chronic diabetic group), but not AAT, for 3-5 weeks. That AAT favorably alters the balance of pro- to anti-inflammatory and enhances local expression of regulatory T cell genes was evident. In pancreatic lymph nodes obtained from AAT treated NODs, we noted dampened expression of the GBP1, PAI-1, and CRP acute phase reactant genes (FIG. 6A). Amplified expression of genes encoding acute phase reactants arises within inflamed tissues. Hence reduced expression of these genes may signify dampened inflammation. In support of this interpretation, reduced expression of pro-inflammatory (IFNγ, IL-6, and IL-1β) cytokine genes was detected within pancreatic lymph nodes obtained from AAT treated NODs (FIG. 6B). Note also that expression of the prototypic Th1-type IFNγ, gene was dampened while expression of the regulatory T cell specific Foxp3 gene was amplified in the pancreatic lymph node of AAT treated diabetic NODs (FIG. 6C). In short, AAT tilted the balance of expression of pro- to anti-inflammatory genes sharply toward predominance of anti-inflammatory gene expression. Similarly, the balance of effector Th1-type to regulatory T cell gene expression events shifted toward immunoregulation. AAT did not alter expression of the SOCS1, SOCS2, TNF-α, and TGF-β genes within the pancreatic lymph node. No additional gene expression events were analyzed.

The AAT Treatment Ablates Insulin Resistance in New Onset T1DM NOD Mice.

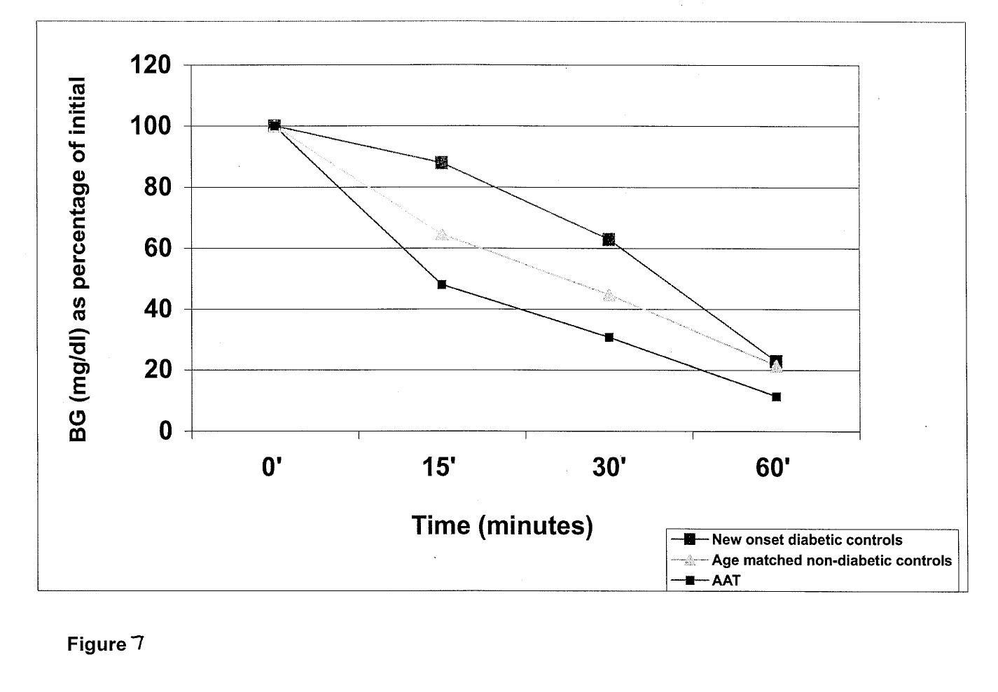

We sought to determine via insulin tolerance tests whether AAT treatment influences the sensitivity of NOD mice to insulin driven disposal of blood glucose. Blood glucose levels in 10 week old new onset diabetic mice fell only 37% over a 1 hr period following an intraperitoneal injection of insulin, but decreased by ca. 80-85% in both AAT treated and age matched control non-diabetic NOD mice (FIG. 7). Thus, AAT treatment ablates insulin resistance, thereby normalizing the response of host tissues to insulin.

AAT Treatment Restores in Vivo Insulin Signaling in Diabetic NOD Mice.

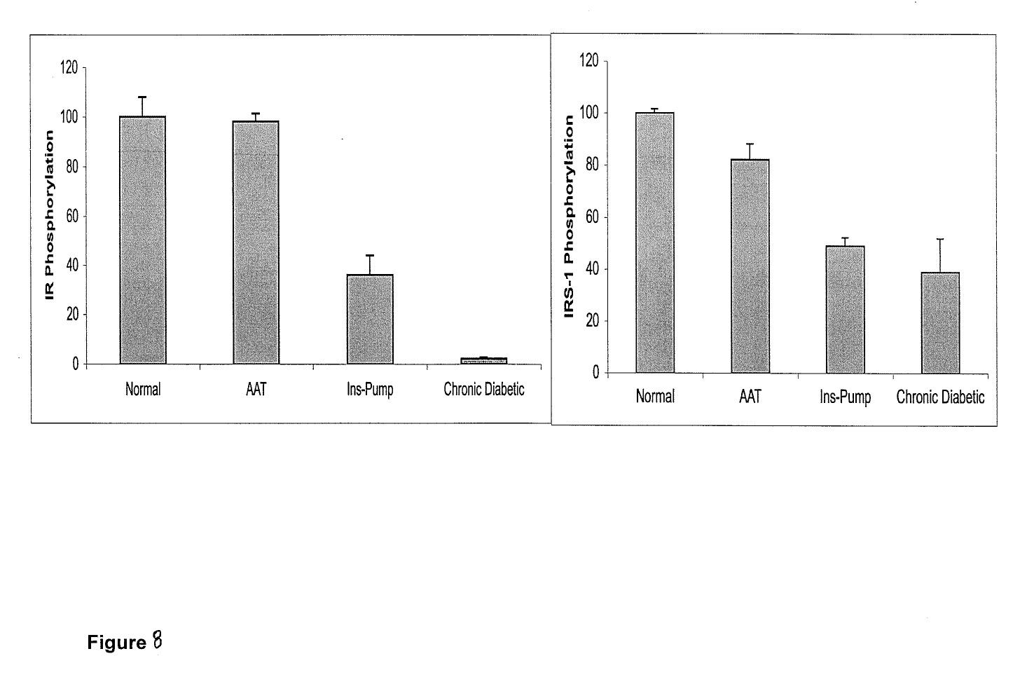

As insulin resistance in new onset diabetic NOD mice is accompanied by defective in vivo insulin signaling in fat and muscle, we examined the effects of AAT upon insulin signaling in skeletal muscle of new onset diabetic NOD mice in vivo. Insulin-stimulated tyrosyl phosphorylation of the insulin receptor (IR) was markedly diminished in new onset T1DM NOD mice, with a 90% reduction in the magnitude of blot densitometry, compared to age matched control non-diabetic NOD mice (FIG. 8). Impaired insulin signaling was also evident with respect to insulin-stimulated tyrosine-phosphorylation of insulin receptor substrate-1 (IRS-1) (FIG. 8) and PKB/Akt (data not shown), molecules that normally transmit the downstream signals of the insulin activated IR (FIG. 8). The impact of short term AAT therapy upon tyrosine phosphorylation patterns in new onset T1DM mice rendered euglycemic by AAT therapy was compared with that obtained with mice rendered euglycemic from the time of diagnosis of T1DM with intense insulin therapy delivered with osmotic pumps. AAT therapy, unlike osmotic insulin pump therapy, does not immediately render the treated mice euglycemic. As AAT treated mice remain hyperglycemic for as long as 3-5 weeks, we temporarily used non-intensive, conventional insulin therapy delivered (i.p.) in AAT treated hosts to prevent extreme hyperglycemia until the advent of euglycemia (at which time insulin therapy is discontinued). As AAT, but not intense osmotic pump delivered insulin or conventional insulin (chronic diabetic group), treatment completely restored the tyrosine-phosphorylation of IR, IRS-1 and PKB/AKT in new onset T1DM NOD mice, AAT treatment apparently ablates insulin resistance (see FIG. 7) via restoration of normal tyrosine phosphorylation dependent insulin signaling (FIG. 8) in new onset T1DM NOD mice.

AAT Treatment Exerts an Anti-Inflammatory Effect in Critical Insulin Sensitive Tissues.

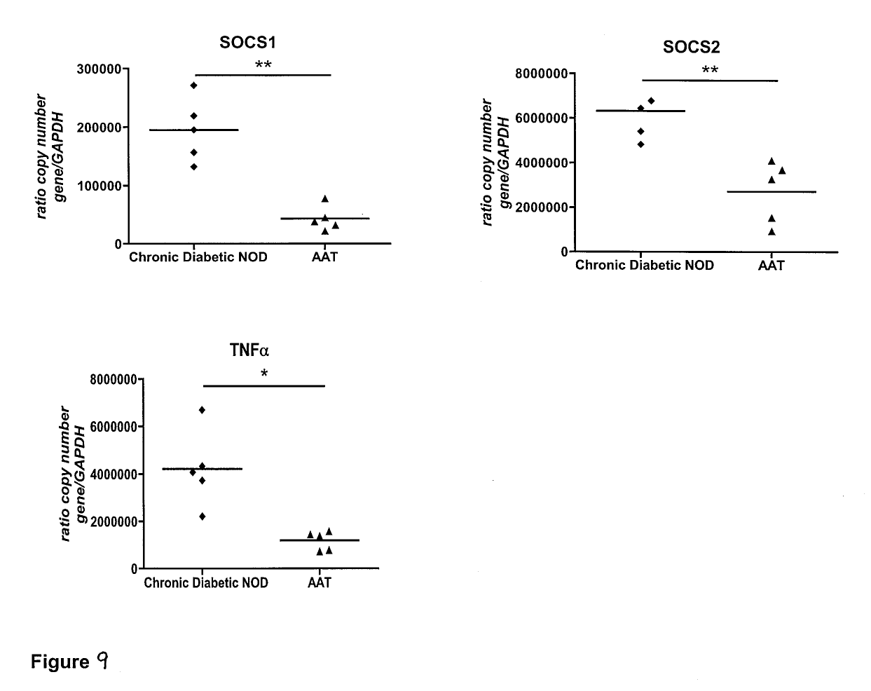

Using RT-PCR methodology, a limited, and hypothesis driven targeted transcriptional profile for select inflammation-associated gene expression events within fat, a key tissue for insulin driven disposal of blood glucose, was compiled in NOD mice (FIG. 9). As AAT treated mice remain hyperglycemic for 3 weeks we temporarily used non-intensive, conventional insulin therapy delivered (i.p.) in AAT treated hosts to prevent extreme hyperglycemia until the advent of euglycemia (at which time insulin therapy is discontinued). Hence, we also analyzed insulin sensitive tissues by RT-PCR in new onset T1DM mice treated by conventional insulin treatment for 3 weeks (chronic diabetic group). Expression of SOCS and TNFα by insulin sensitive tissues creates insensitivity to insulin driven disposal of blood glucose (Hotamisligil, Nature, 444:860-867, 2006; Shoelson et al., J. Clin. Inv., 116:1793-1801, 2006). Hence, we analyzed the expression of TNF-α and SOCS 1, and 2 in fat of AAT treated and control T1DM NOD mice. As compared to control chronic diabetic NODs, expression of TNFα as well as the SOCS 1 and 2 genes was markedly reduced in AAT treated diabetic mice (FIG. 9). Hence restoration of euglycemia, normal insulin sensitivity and in vivo insulin signaling by AAT treatment is linked to reduced expression of pro-inflammatory molecules known to impair insulin responsiveness in tissues critical for insulin triggered disposal of blood glucose.

The NOD model of autoimmune mediated diabetes shares many features, including common susceptibility genes and a similar pattern of T cell dependent anti-beta cell immunity, with human Type 1 diabetes. (Rossini et al., Annu. Rev. Immunol., 3:289-320, 1985; Shoda et al., Immunity, 23:115-126, 2005). In the NOD model, the loss of immune tolerance to beta cells creates vulnerability to autoimmune mediated destruction of insulin producing beta cells within the islets of Langerhans. It is notable that few T cell directed therapies have succeeded in restoring euglycemia and self-tolerance to islets in overtly diabetic NOD mice (Belghith et al., Nature Med., 9:1202-1208, 2003; Ogawa et al., Diabetes, 53:1700-1705, 2004; Tarbell et al., J. Exp. Med., 204:191-201, 2007). We suspect that an inability of many T cell directed treatments to quench and control pro-inflammatory responses, responses that are not directly mediated by T cells, results in the failure of these T cell directed treatments to restore euglycemia and immune tolerance to beta cells. To directly test this hypothesis, we treated new onset overtly diabetic mice with a short course of human AAT, an acute phase reactant with proteinase, anti-inflammatory, anti-leukocyte migratory and anti-apoptotic effects (Breit et al., Clin. Immunol. Immunopathol. 35:363-380, 1985; Churg et al., Lab. Inv. 81:1119-1131, 2001; Jie et al., Chinese Med. J. 116:1678-1682, 2003; Lewis et al., Proc. Natl. Acad. USA 102:12153-12158, 2005; Petrache et al., Am. J. Resp. Crit. Care Med. 173:1222-1228, 2006). As expression of AAT, a potent anti-inflammatory protein, sharply rises in response to inflammation, it seems reasonable to speculate that the function of this protein is to limit the duration, magnitude and perhaps molecular texture of inflammation (Brantly, Am. J. Resp. Cell Mol. Biol. 27:652-654, 2002).

Despite the absence of direct action upon T cell activation, human AAT therapy induces tolerance to allogeneic islet transplants. (Lewis et al., Nat. Immunol. 2007; Arora et al., Nature 274:589-590, 1978; Lewis et al., Proc. Natl. Acad. Sci. USA 102:12153-12158, 2005). As we demonstrate herein, human AAT therapy, despite the known immunogenicity of human AAT in mice, quickly halts invasive and cytodestructive insulitis type autoimmunity in the NOD model. Both euglycemia and immune tolerance to beta cells are restored. The ability of AAT therapy to modify the inflammatory context in which autoantigen is recognized by T cells may play an important role in quenching destructive autoimmunity. The cytokine texture of the environment in which naïve CD4+ T cells recognize antigen dictates the commitment of these cells to various effector (Th1, Th2, Th17) or Foxp3+ regulatory phenotypes. (Bettelli et al., Nature 441:235-238, 2006; Veldhoen et al., Immunity 24:179-189, 2006; Tato et al., Nature 441:166-168, 2006). For example, IL-12 spurs commitment to the Th1 phenotype, TGF-β triggers commitment to the regulatory T cell phenotype. The concurrent presence of TGF-β and IL-6 fosters commitment to the Th17 phenotype and reciprocally blocks commitment to the FOXP3+ regulatory T cell phenotype (Bettelli et al., Nature 441:235-238, 2006; Veldhoen et al., Immunity 24:179-189, 2006; Tato et al., Nature 441:166-168, 2006). Following AAT therapy an islet invasive form of insulitis was supplanted by a circumferential type of insulitis that is often associated with tolerance to islets. (Rossini et al., Annu. Rev. Immunol. 3:289-320, 1985; Shoda et al., Immunity 23:115-126, 2005). The rapid ablation of invasive insulitis and the marked decrease in pro-inflammatory cytokine and reciprocal rise in Foxp3 gene expression within the pancreatic lymph node suggest that AAT triggered alterations in inflammation can rapidly alter the vigor and fundamental nature of T cell dependent autoimmunity. Moreover, AAT treated NOD mice are tolerant to syngeneic islets. In short, a marked decrease in expression of pro-inflammatory cytokines is associated with and probably causal for restoration of immune tolerance to islets.

Importantly, the advent of overt hyperglycemia occurs before the complete loss of beta cells and restoration of euglycemia occurs in AAT treated diabetic NODs occurs very quickly. Recently an insulin resistant state, more classically noted as a feature of Type 2 diabetes mellitus (T2DM), has been discovered in new onset, overtly diabetic T1DM NOD mice (Chaparro et al., Proc. Natl. Acad. USA 103:12475-12480, 2006). Under normal conditions, insulin stimulates disposal of blood glucose into muscle, fat and, to a lesser extent, into other insulin sensitive tissues. A molecular hallmark of insulin driven glucose disposal is the insulin triggered tyrosyl phosphorylation of insulin receptor and immediate downstream signaling molecules within critical insulin sensitive tissues (e.g. fat and muscle). In obesity related T2DM, a deficiency in insulin driven glucose disposal is accompanied by and probably arises as a consequence of faulty phosphorylation of the insulin receptor (Hotamisligil, Nature 444:860-867, 2006; Shoelson et al., J. Clin. Inv. 116:1793-1801, 2006). The proximal cause of the insulin resistance and linked faulty tyrosyl phosphorylation of the insulin receptor in obesity linked T2DM state is inflammation of critical insulin sensitive tissues (Reviewed in Hotamisligil, Nature 444:860-867, 2006; Chaparro et al., Proc. Natl. Acad. Sci. USA 103:12475-12480, 2006). In fact, we note both insulin resistance and gross hypo-phosphorylation of the insulin receptor in new onset T1DM NOD mice. Both insulin resistance and hypo-phosphorylation of the insulin receptor were corrected in parallel by AAT treatment. In contrast restoration of euglycemia with intense insulin treatment did not produce a remission in insulin resistance or the hypophosphorylation of the insulin activated insulin receptor. These data suggest that the prompt relief from hyperglycemia induced by AAT treatment was linked, at least in part, to resolution of both the faulty insulin signaling and insulin resistance. This situation also pertained to our observations with a curative triple therapy regimen consisting of rapamycin+IL-2/Fc+mutant IL-15/Fc. Hence it seems likely that the ability of both AAT and the triple therapy regimen to relieve faulty insulin signaling and insulin resistance is linked to their ability to restore euglycemia. The presence of both Type1-like autoimmune beta cell destruction and Type 2-like insulin resistance may suggest that two separate disease processes converge to create hyperglycemia before the advent of total beta cell destruction in the NOD model. We now present evidence indicating that an inflammatory state is the likely proximal cause of both autoimmunity and insulin resistance. Indeed, the restoration of euglycemia and immune tolerance to beta cells in parallel with relief from progressive, destructive beta cell directed autoimmunity and from insulin resistance can be accomplished through application of AAT, an acute phase reactant protein that modifies a pro-inflammatory state evident in overtly diabetic NOD mice. Hence, we propose that a pro-inflammatory state present in NOD mice triggers both autoimmunity and insulin resistance. Note that, the beta cell mass expanded markedly and rapidly in AAT treated hosts. These findings tend to further authenticate the potent AAT fostered cytoprotective effects upon islets demonstrated by Lewis et al. (Lewis et al., Proc. Natl. Acad. USA 102:12153-12158, 2005; Lewis et al., Nat. Immunol. 2007). Indeed this is the first demonstration in which successful treatment of new onset diabetic mice in the absence of islet cell transplantation routinely leads to expansion of the beta cell mass.

Successful application of therapies that restore euglycemia in overtly diabetic NOD mice has predictive value for human T1DM (Shoda et al., Immunity 23:115-126, 2005). The excellent results achieved with anti-CD3 treatment in diabetic NOD mice have served as the basis for initiating successful clinical trials of anti-CD3 mAb in humans with T1DM. (Herold et al., New England J. Med. 346:1692-1698, 2002). Indeed, anti-CD3 mAb treatment slows the progression to permanent diabetes in humans with new onset T1DM. (Herold et al., New England J. Med. 346:1692-1698, 2002; Keymeulen et al., New England J. Med. 352:2598-2608, 2005).

Materials and Methods:

T cell activation study: Single-cell suspensions of purified T cells C57BL/6 were prepared from spleen and lymph node and labelled with the vital dye carboxyfluorescein diacetate succinmidyl ester (CFSE) (Molecular Probes-Invitrogen, Carlsbad, Calif.). (Auchincloss et al., Proc. Natl. Acad. Sci. USA, 90:3373-3377, 1993). The cells were cultured at 1×10⁵ cells/well in 96-well flat-bottom plates coated with anti-CD3 mAb (eBioscience, San Diego, Calif.; 2.5 ug/mL) and soluble anti-CD28 mAb (eBioscience; 2.5 ug/mL), in a final volume of 250 μL of complete medium at 37° C. in 5% CO2 for four days (Bettelli et al., Nature, 441:235-238, 2006), in the presence or in the absence of AAT (0.5 ug/mL). CFSE profile was used to assess the proliferation of the responder population by gating onto the CD3⁺ population. Cells were counterstained with CD25-PE, CD44-PE or CD62L-PE (eBioscience) in order to determine the expression of T cell activation proteins.

Mice: Female NOD (NOD/LtJx) mice and NOD.SCID (NOD.CB17-Prkdc^{scid}/J) were purchased from Jackson Laboratories (Bar Harbor, Me.) at 4 weeks of age and maintained under pathogen-free conditions at the Massachusetts General Hospital (Boston, Mass.).

Blood glucose levels of NOD mice were monitored twice weekly with the Accu-Check blood glucose monitor system (Roche, Indianapolis, Ind.). When non-fasting blood glucose levels are in excess of 300 mg/dl on three consecutive measurements, a diagnosis of new onset of diabetes is made. For syngeneic islet transplant recipients, blood glucose levels were checked at the time of transplantation, then daily for two weeks, and then 2 to 3 times per week afterward.

Induction of and management of diabetes: Successfully AAT treated euglycemic NOD mice were rendered hyperglycemic with stz (275 mg/kg i.p) treatment 200 to 300 days following the restoration of euglycemia in treated and formerly spontaneously diabetic NOD. With the re-emergence of hyperglycemia following stz administration, these diabetic NOD mice were used as syngeneic or allogeneic islets graft recipients. Graft failure was defined as the first day of 3 consecutive days of blood glucose levels >250 mg/dl.

Islet transplantation: NOD.SCID mice and C57BL/6 mice (10-12 weeks old) were used as donors for islet transplants. Islets were isolated using a modification of the method of Gotoh et al. (Transplantation 40:437, 1985), in which the pancreatic duct is distended with collagenase P. After Histopaque gradient (Histopaque^{R}-1077, Sigma Chemical Co., St. Louis, Mo.) purification, islets with diameters between 75 and 250 μm were hand picked and transplanted under the renal capsule. Each recipient received 600-800 NOD.SCID or C57BL/6 islets.

Alpha1 antitrypsin treatment protocol: Aralast™ (human α-proteinase inhibitor) is a major serum serine-protease inhibitor which inhibits the enzymatic activity of neutrophil elastase, cathespin G, proteinase 3, thrombin, trypsin and chymotrypsin. Aralast was purchased from Baxter (Westlake Village, Calif.) and was given at a dose of 2 mg i.p every 3 days for a total of 5 injections.

Quantitative real-time PCR Methods: Messenger RNA was extracted from fat and muscle using Invitrogen's Micro to Midi kit (Carlsbad, Calif.) according to the manufacturer's protocol. Reverse transcription was carried out with 1 μg of RNA using ABI Prism TaqMan reverse transcription reagents (Foster City, Calif.) with random hexamers as primers. (Li et al. New. Eng. J. Med. 344:947-954, 2001). Oligonucleotide primers and fluorogenic probes (FAM) were designed and synthetized for the measurement of mRNA levels of Suppressor of cytokine signaling1 (SOCS1), Suppressor of cytokine signaling2 (SOCS2), Suppressor of cytokine signaling3 (SOCS3), tissue necrosis factor alpha. (TNFα), Complement 3 (C3), C-reactive protein (CRP), Guanylate nucleotide binding protein-1 (GBP1), interleukin-1 beta (IL-1β), interferon gamma (IFNγ), plasminogen activator inhibitor type-1 (PAI-1), and Foxp3. Quality controls were performed to validate their specificify and their real-time PCR efficiency. PCR analysis was performed by a two-step process. In the first step, a pre-amplification reaction was set up using the ABI bio-systems thermo cycler (10 cycles) with 3 μl cDNA and 7 μl of dNTP, 10× PCR buffer, Taq DNA polymerase, and gene specific oligonucleotide primer pairs. This was followed by measurement of transcripts with an ABI Prism 7900HT sequence detection system. The PCR amplicon for 18S rRNA was kindly provided by Dr. Suthanthiran, Weill Medical College of Cornell University, New York, USA. 18S rRNA amplicon was quantified and used for developing standard curves. Messenger RNA levels in the samples were normalized to the expression of GAPDH and Transcript levels were calculated according to the absolute quantification method (Ding et al. Transplantation 75:1307-1312, 2003), as described by the manufacturer.

Insulin tolerance tests, morpohometric analyses of beta cell mass, in vivo insulin signaling studies, and immunoblotting were performed as described in Example 1.

A number of embodiments of the invention have been described. Nevertheless, it will be understood that various modifications may be made without departing from the spirit and scope of the invention. Accordingly, other embodiments are within the scope of the following claims.

What is claimed is:

1. A pharmaceutical composition comprising:
a first agent that selectively stimulates regulatory T cells or selectively inhibits inflammatory T cells; and
a second agent that reduces an inflammatory response in a tissue of a patient to whom the composition is administered, wherein the second agent reduces the expression or activity of a pro-inflammatory cytokine, promotes the expression or activity of an anti-inflammatory cytokine, or both.

2. The pharmaceutical composition of claim 1, wherein the composition is formulated for intravenous, intramuscular, or subcutaneous administration.

3. The pharmaceutical composition of claim 1 or claim 2, wherein the first agent is:
(a) rapamycin;
(b) an anti-CD3 antibody or antigen binding fragment thereof;
(c) a non-lytic anti-CD4 antibody or antigen binding fragment thereof;
(d) a T cell immunoglobulin mucin 3 (TIM3) agonist;
(e) a T cell immunoglobulin mucin 1 (TIM1) antagonist;
(f) galectin 9 and agonists thereof;
(g) an agent that selectively inhibits Th17 cells;
(h) an agent that inhibits the expression or activity of interleukin 17 (IL-17);
(i) an IL-15 antagonist;
(j) an IL-2 agonist; or
(i) a combination thereof.

4. The pharmaceutical composition of any of claim 3, wherein the IL-15 antagonist comprises a mutant IL-15 polypeptide that binds to an IL-15 receptor (IL-15R) but fails to fully activate signal transduction through the IL-15R.

5. The pharmaceutical composition of any of claims 1-4, wherein the second agent is:
(a) α1-antitrypsin or an agent that promotes the expression or activity of α1-antitrypsin;
(b) an adenosine agonist;
(c) an agent that induces expression or activity of heme oxygenase-1 (HO-1);
(d) immunoregulatory antigen presenting cells (APC) or regulatory T cells;
(e) an adenylate cyclase activator;
(f) a cytokine selected from the group consisting of IL-1rn, IL-4, IL-10, IL-11, IL-13, and TGF-β, or an agent that promotes the expression or activity of IL-1rn IL-4, IL-10, IL-11, IL-13, or TGF-β;
(g) an agent that inhibits the expression or activity of one of the following cytokines: TNF-α, IFN-γ, GM-CSF, MIP-2, IL-6, IL-12, IL-1α, IL-1β, IL-21, and IL-23;
(h) Vitamin D or an analogue thereof; or
(i) or a combination thereof.

6. The composition of claim 5, wherein the adenylate cyclase activator is a prostaglandin.

7. The composition of any of claims 1-5, wherein the first agent comprises an IL-15 antagonist, an IL-2 agonist, and/or rapamycin.

8. The composition of any of claims 1-5, wherein the second agent comprises α1-antitrypsin or an agent that promotes the expression or activity of α1-antitrypsin.

9. The composition of any of claim 1-5, wherein the second agent is an agent that inhibits the expression or activity of one of the following cytokines: TNF-α, IFN-γ, GM-CSF, MIP-2, IL-6, IL-12, IL-1α, IL-1β, IL-21, and IL-23; and wherein the agent comprises a receptor of the cytokine

10. The composition of claim 9, wherein the agent further comprises the Fc region of an immunoglobulin.

11. The composition of any of claim 1-5, wherein the second agent is an agent that inhibits the expression or activity of one of the following cytokines: TNF-α, IFN-γ, GM-CSF, MIP-2, IL-6, IL-12, IL-1α, IL-1β, IL-21, and IL-23; and wherein the agent comprises an antibody or antigen binding fragment thereof that specifically binds to the cytokine.

12. The composition of any of claims 1-5, wherein the second agent comprises IL-10 or an agent that promotes the expression or activity of IL-10.

13. The composition of claim 12, wherein the IL-10 is fused to a heterologous molecule that increases the circulating half-life of the IL-10.

14. The composition of claim 13, wherein the heterologous molecule comprises the Fc region of an immunoglobulin.

15. A method of treating a patient at risk for, or diagnosed as having, an autoimmune disorder, the method comprising administering to the patient an effective amount of the composition of any of claims 1-14.

16. The method of claim 15, wherein the patient has been diagnosed as having Type 1 diabetes.

17. A method of treating a patient who is insulin resistant, the method comprising administering to the patient an effective amount of the composition of any of claims 1-14.

18. The method of claim 17, wherein the patient has Type 2 diabetes, is at risk of developing Type 2 diabetes, has metabolic syndrome, or has Type 1 diabetes.

19. A method of treating a patient who has received a transplant of an organ, tissue, or cells, or who is scheduled to receive a transplant of an organ, tissue, or cells, the method comprising administering to the patient an effective amount of the composition of any of claims 1-14.


SEQUENCE LISTING (2 sequences)

SEQ ID NO: 1 - DNA, 1607 bp, Homo sapiens
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 2 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK